(19) United States
(12) Patent Application Publication
(10) Pub. No.: US 2025/0381271 A1
(43) Pub. Date: Dec. 18, 2025

(54) CELL SYSTEM AND APPLICATION THEREOF, AND METHOD FOR ACTIVATING BROAD-SPECTRUM CANCER CELL-SPECIFIC T CELL

(71) Applicant: SUZHOU ERSHENG BIOMEDICAL CO., LTD., Suzhou, Jiangsu (CN)

(72) Inventor: Mi LIU, Suzhou (CN)

(73) Assignee: SUZHOU ERSHENG BIOMEDICAL CO., LTD., Suzhou, Jiangsu (CN)

(21) Appl. No.: 18/860,898

(22) PCT Filed: May 26, 2022
(86) PCT No.: PCT/CN2022/095265

§ 371 Date: Oct. 28, 2024

(30) Foreign Application Priority Data
Apr. 29, 2022 (CN) 202210468876.3

(51) Int. Cl. A61K 40/11; A61K 35/17; A61K 40/42; A61K 40/50; A61P 35/00; C12N 5/0783

(52) U.S. Cl. CPC A61K 40/11; A61K 35/17; A61K 40/42; A61K 40/50; A61P 35/00; C12N 5/0636; A61K 2239/54; C12N 2501/05; C12N 2501/22; C12N 2501/23; C12N 2501/24; C12N 2501/25; C12N 2502/1107; C12N 2502/1121; C12N 2502/1157

(57) ABSTRACT

A cell system and an application thereof, and a method for activating a broad-spectrum cancer cell-specific T cell. The cell system includes a cancer cell-specific T cell extracted from a tumor-infiltrating lymphocyte, where the extraction includes steps of co-incubating the tumor-infiltrating lymphocyte or a T cell in the tumor-infiltrating lymphocyte and an antigen-presenting cell with a nanoparticle and/or a microparticle loaded with a whole-cell antigen of a cancer cell to activate a cancer cell-specific T cell, and then isolating the activated cancer cell-specific T cell from the tumor-infiltrating lymphocyte. The problem that broad-spectrum and polyclonal cancer cell-specific T cells in tumor-infiltrating lymphocytes cannot be effectively screened in clinical practice at present is overcome, broad-spectrum effector cancer cell-specific T cells with a specific tumor-killing function can be isolated from the tumor-infiltrating lymphocytes, which have the characteristics of easy isolation and high specificity, and can be used for cancer prevention and treatment.

### CROSS-REFERENCE TO RELATED APPLICATIONS

The present disclosure is a National Stage of International Application No. PCT/CN2022/095265, filed on May 26, 2022, which claims priority to the Chinese Patent Application No. 202210468876.3, filed on Apr. 29, 2022, both of which are hereby incorporated by reference in their entireties.

### REFERENCE TO ELECTRONIC SEQUENCE LISTING

This application includes an electronically submitted sequence listing in .txt format. The .txt file contains a sequence listing entitled “7166-0223PUS1_ST25.txt” created on Aug. 22, 2025 and is 2,162 bytes in size. The sequence listing contained in this .txt file is part of the specification and is hereby incorporated by reference herein in its entirety.

### TECHNICAL FIELD

The present disclosure relates to the field of immunotherapy, and in particular to a cell system and an application thereof, and a method for activating a broad-spectrum cancer cell-specific T cell.

### BACKGROUND

Tumor-infiltrating lymphocytes (TILs) are a type of infiltrating lymphocytes isolated from tumor tissue, mainly represented by T cells, B cells, macrophages, and NK cells. They are an important component of the tumor microenvironment and play a central role in the immune response to tumors, significantly affecting the treatment and prognosis of cancer patients. Among TILs, T cells, especially cancer cell-specific T cells, play the role of the main force in anti-cancer. T cells are the main cells in the body that specifically recognize and kill cancer cells. Each clone of cancer cell-specific T cells may specifically recognize an antigen epitope. Cancer patients, especially those who have undergone immunotherapy or radiotherapy, contain a certain number of cancer cell-specific T cells in their bodies.

Research has shown that the more lymphocytes, especially cancer cell-specific T cells, that can infiltrate into the tumor site, the better the tumor tissue can be controlled. However, different types of TILs play different roles in various tumor subtypes. Among TILs, there are not only cells that positively regulate and exert immune surveillance, such as CD8⁺ TIL, NK cells, CD4⁺ Th1 cells, etc., but also cells that negatively regulate and exert immune tolerance, such as CD4⁺ Th2 cells and regulatory T cells (Treg). Therefore, many lymphocytes that infiltrate into the tumor site do not necessarily exert anti-cancer effects. For example, type 2 macrophages can promote tumor growth instead, and regulatory T cells, especially cancer-specific regulatory T cells, can also promote cancer growth.

TILs in tumor patients are inhibited for various reasons, including the above factors, and cannot effectively kill tumors. Therefore, people hope to enrich TIL cells through some in vitro culture methods and then infuse them back into patients to exert anti-tumor effects, that is, TIL cell therapy. The therapy of using tumor-infiltrating lymphocytes (TILs) to treat cancer has been developed for many years, but still faces the problem of how to better screen killer cancer cell-specific T cells (especially the problem of sorting broad-spectrum cancer cell-specific T cells). The current method mainly involves isolating T cells and other cells from treated tumor tissue for in vitro expansion and back infusion to patients for use. However, many tumor-infiltrating T cells are not all tumor-specific T cells, and many tumor-specific T cells are regulatory T cells (Treg) or exhausted T cells, which cannot effectively recognize and kill cancer cells. Therefore, even if T cells are isolated and expanded from the body and then infused back into patients, the effect is relatively limited. Moreover, if Treg is expanded in vitro and then infused back into patients, it will cause faster growth of tumor tissue instead. Therefore, how to screen effector cancer cell-specific T cells with cancer cell recognition and killing functions among tumor-specific T cells from tumor-infiltrating lymphocytes is very critical. However, there is currently no particularly efficient means that may be used to isolate this part of effector cancer cell-specific T cells with specific tumor-killing functions from tumor-infiltrating lymphocytes in a good manner.

### SUMMARY

To solve the above technical problems, the present disclosure provides a cell system derived from a tumor-infiltrating lymphocyte, which uses a nanoparticle or a microparticle loaded with a whole-cell antigen of a cancer cell to first activate an effector cancer cell-specific T cell in vitro, and then utilizes a marker specifically expressed by an activated effector cancer cell-specific T cell to isolate and extract the above cancer cell-specific T cell, and infused back into a patient to prevent or treat cancer, effectively solving the problem of how to specifically isolate and extract broad-spectrum and polyclonal cancer cell-specific T cells with the ability of recognizing and killing cancer cells from tumor-infiltrating lymphocytes.

A first purpose of the present disclosure is to provide a cell system derived from a tumor-infiltrating lymphocyte, the cell system including a cancer cell-specific T cell extracted from the tumor-infiltrating lymphocyte, where the extraction includes steps of co-incubating (1) the tumor-infiltrating lymphocyte or a T cell in the tumor-infiltrating lymphocyte and (2) an antigen-presenting cell with (3) a nanoparticle (NP) or a microparticle (MP) loaded with a whole-cell antigen of a cancer cell loaded with the whole-cell antigen of the cancer cell to activate the cancer cell-specific T cell, and then isolating the cancer cell-specific T cell activated by the whole-cell antigen of the cancer cell, where the whole-cell antigen of the cancer cell includes a water-soluble antigen and/or a water-insoluble antigen obtained by lysing the cancer cell and/or tumor tissue, and the water-insoluble antigen is loaded onto the nanoparticle or microparticle after being solubilized by a solubilizing agent or a solubilizing solution containing a solubilizing agent.

Furthermore, after isolating the cancer cell-specific T cell, a step of expanding the cancer cell-specific T cell or a step of expanding and sorting the cancer cell-specific T cell is further included.

Furthermore, the expanding is in vitro expanding, and a method of the expanding and sorting includes but is not limited to co-incubating with a cytokine and/or an antibody.

Furthermore, the cytokine includes but is not limited to interleukin 2 (IL-2), interleukin 7 (IL-7), interleukin 14 (IL-14), interleukin 4 (IL-4), interleukin 15 (IL-15), interleukin 21 (IL-21), interleukin 17 (IL-17), interleukin 12 (IL-12), interleukin 6 (IL-6), interleukin 33 (IL-33), interferon-γ (IFN-γ), and TNF-α.

Furthermore, the antibody includes but is not limited to an αCD-3 antibody, an αCD-4 antibody, an αCD-8 antibody, an αCD-28 antibody, an αCD-40 antibody, an αOX-40 antibody, and an αOX-40L antibody.

Furthermore, the obtained cancer cell-specific T cell includes a CD4⁺ T cell and/or a CD8⁺ T cell, and preferably including both a CD4⁺ T cell and a CD8⁺ T cell.

Furthermore, the isolating includes a step of screening using a specific surface marker of the cancer cell-specific T cell activated by the whole-cell antigen of the cancer cell. The specific surface marker includes but is not limited to CD69, CD25, OX40 (CD134), CD137, CD28, etc.

Furthermore, a technique for isolating the cancer cell-specific T cell using the surface marker includes, but is not limited to, flow cytometry and magnetic bead sorting.

In the present disclosure, the nanoparticle and/or the microparticle loaded with the whole-cell antigen of the cancer cell are used to specifically activate the cancer cell-specific T cell pre-stored in the tumor-infiltrating lymphocyte that has already been activated in a lymph node. Then utilizing the characteristic of the activated cancer cell-specific T cell secreting a specific cytokine or highly expressing certain surface molecules, the cancer cell-specific T cell is isolated and obtained by flow cytometry and other means, which is infused back into the patient after in vitro expansion, and can be isolated and expanded to the most diverse and broad-spectrum cancer cell-specific T cell with the function of recognizing and killing the cancer cell.

Furthermore, the co-incubating includes, but is not limited to: simultaneously co-incubating the nanoparticle and/or the microparticle loaded with the whole-cell antigen of the cancer cell, the antigen-presenting cell, and the tumor-infiltrating lymphocyte or the T cell in the tumor-infiltrating lymphocyte; or first co-incubating the nanoparticle and/or the microparticle loaded with the whole-cell antigen of the cancer cell with the antigen-presenting cell for a period of time, and then adding the tumor-infiltrating lymphocyte or the T cell in the tumor-infiltrating lymphocyte for simultaneous co-incubation; or first co-incubating the nanoparticle and/or the microparticle loaded with the whole-cell antigen of the cancer cell with the antigen-presenting cell for a period of time, isolating the antigen-presenting cell, and simultaneously co-incubating the antigen-presenting cell with the tumor-infiltrating lymphocyte or the T cell in the tumor-infiltrating lymphocyte.

Furthermore, the above T cell in the tumor-infiltrating lymphocyte is a T cell sorted from the tumor-infiltrating lymphocyte, the sorted T cell includes a cancer cell-specific T cell, and a method for sorting the T cell from the tumor-infiltrating lymphocyte may be flow cytometry, magnetic bead sorting, etc. Specifically, a CD45⁺ cell and/or a CD3⁺ cell, a CD45⁺ CD3⁺ cell, a CD3⁺ CD8⁺ cell, a CD45⁺ CD3⁺ CD8⁺ cell, a CD3⁺ CD4⁺ cell, or a CD45⁺ CD3⁺ CD4⁺ cell is sorted from the tumor-infiltrating lymphocyte using flow cytometry or magnetic bead sorting.

Furthermore, the above tumor-infiltrating lymphocyte or the T cell in the tumor-infiltrating lymphocyte may not be treated, or the cell-derived body can be treated by radiotherapy, immunotherapy, chemotherapy, particle therapy, vaccine therapy, etc.

Furthermore, the tumor-infiltrating lymphocyte or the T cell in the tumor-infiltrating lymphocyte is derived from an autologous or allogeneic source.

Furthermore, the antigen-presenting cell includes at least one of a B cell, a dendritic cell (DC) and a macrophage, and preferably two or more, such as a B cell and a DC cell.

Furthermore, the antigen-presenting cell may be derived from an autologous or allogeneic source with the tumor-infiltrating lymphocyte or the T cell in the tumor-infiltrating lymphocyte, or may be transformed from a cell line or from a stem cell. It may be known by those skilled in the art that the antigen-presenting cell may be derived from any method by which a peripheral immune cell may be prepared and isolated.

Furthermore, the nanoparticle and/or the microparticle loaded with the whole-cell antigen of the cancer cell are co-incubated with the antigen-presenting cell and the tumor-infiltrating lymphocyte or the T cell in the tumor-infiltrating lymphocyte for at least 4 hours, so that the antigen can be delivered into the antigen-presenting cell and may be processed and presented to a surface of the antigen-presenting cell by the antigen-presenting cell. In the following embodiments, the time for the co-incubating is at least 4 hours, and preferably 24-96 hours.

Furthermore, when the nanoparticle and/or the microparticle loaded with the whole-cell antigen of the cancer cell are co-incubated with the antigen-presenting cell and the tumor-infiltrating lymphocyte or the T cell, a cytokine may be added to the system; the cytokine includes but is not limited to an interleukin, an interferon, a colony-stimulating factor, and a tumor necrosis factor; and the interleukin includes but is not limited to interleukin 2 (IL-2), interleukin 7 (IL-7), interleukin 14 (IL-14), interleukin 4 (IL-4), interleukin 15 (IL-15), interleukin 21 (IL-21), interleukin 17 (IL-17), interleukin 12 (IL-12), interleukin 6 (IL-6), and interleukin 33 (IL-33).

Furthermore, the cancer cell or the tumor tissue is frozen at −20° C. to 273° C., and subjected to repeated freeze-thaw lysis with water or a solution containing no solubilizing agent to obtain a supernatant which is a water-soluble antigen, and a portion of the precipitate which is solubilized by the solubilizing agent and becomes soluble is a water-insoluble antigen.

Furthermore, the water-soluble antigen and/or the water-insoluble antigen is loaded inside the particle and/or loaded on the surface of the particle. Specifically, a way of the loading is that the water-soluble antigen and the water-insoluble antigen of the cell are respectively or simultaneously encapsulated in the particle and/or respectively or simultaneously loaded on the surface of the particle, including but not limited to the water-soluble antigen being simultaneously loaded in the particle and on the surface of the particle, the water-insoluble antigen being simultaneously loaded in the particle and loaded on the surface of the particle, the water-soluble antigen being loaded in the particle but the water-insoluble antigen being loaded on the surface of the particle, the water-insoluble antigen being loaded in the particle and the water-soluble antigen being loaded on the surface of the particle, the water-soluble antigen and the water-insoluble antigen being loaded in the particle but only the water-insoluble antigen being loaded on the surface of the particle, the water-soluble antigen and the water-insoluble antigen being loaded in the particle but only the water-soluble antigen being loaded on the surface of the particle, the water-soluble antigen being loaded in the particle but the water-soluble antigen and the water-insoluble antigen being simultaneously loaded on the surface of the particle, the water-insoluble antigen being loaded in the particle, but the water-soluble antigen and the water-insoluble antigen being simultaneously loaded on the surface of the particle, and the water-soluble antigen and the water-insoluble antigen being simultaneously loaded in the particle and the water-soluble antigen and the water-insoluble antigen being simultaneously loaded on the surface of the particle.

Furthermore, the nanoparticle or the microparticle is further loaded with an immune-enhancing adjuvant. The immune-enhancing adjuvant includes but is not limited to at least one of a microorganism-derived immune enhancer, a product of the human or animal immune system, an intrinsic immune agonist, an adaptive immune agonist, a chemically synthesized drug, fungal polysaccharides, traditional Chinese medicine, and others; and the immune-enhancing adjuvant includes but is not limited to at least one of the active ingredients of a pattern recognition receptor agonist, Bacillus Calmette-Guerin (BCG), a manganese-related adjuvant, a BCG cell wall backbone, a BCG methanol extract residue, BCG muramyl dipeptide, Mycobacterium phlei, polyactin, mineral oil, a virus-like particle, an immune-enhanced reconstituted influenza virosome, a cholera enterotoxin, a saponin and a derivative thereof, resiquimod, thymosin, a neonatal bovine liver active peptide, miquimod, a polysaccharide, curcumin, immune adjuvant CpG, immune adjuvant poly(I:C), immune adjuvant poly ICLC, Corynebacterium pumilus vaccine, a hemolytic streptococcus preparation, coenzyme Q10, levamisole, polycytidylic acid, a manganese adjuvant, an aluminum adjuvant, a calcium adjuvant, various cytokines, an interleukin, an interferon, polyinosinic acid, polyadenylic acid, alum, aluminum phosphate, lanolin, squalene, a cytokine, vegetable oil, endotoxin, a liposome adjuvant, MF59, double-stranded RNA, double-stranded DNA, an aluminum-related adjuvant, CAF01, ginseng, and Astragalus membranaceus. It may be understood by those skilled in the art that the enumerated herein is not exhaustive, and other substances that may enhance the immune response may also be adopted as the immune-enhancing adjuvant.

Preferably, the immune-enhancing adjuvant is a Toll-like receptor agonist; more preferably, two or more Toll-like receptor agonists are used in combination to ensure that the nanoparticle or the microparticle may better activate the cancer cell-specific T cell after being phagocytosed by the antigen-presenting cell.

Furthermore, the use of two or more Toll-like receptor agonists in combination is the use of poly(I:C)/Poly(ICLC) and CpG-ODN (CpG oligodeoxynucleotide) in combination. Preferably, the CpG-ODN is two or more types of CpG-ODNs.

Furthermore, the adjuvant may be loaded inside and/or on the surface of the nanoparticle or the microparticle.

Furthermore, the nanoparticle or the microparticle loaded with the whole-cell antigen of the cancer cell is further simultaneously co-loaded with a substance that increases lysosome escape.

Furthermore, the substance that increases lysosome escape includes but is not limited to a carrier and a material that increases osmotic pressure within a lysosome, a carrier and a material that decreases the stability of a lysosomal membrane, and a substance with a proton sponge effect, which may be loaded inside and/or on the surface of the nanoparticle or the microparticle.

Furthermore, the substance that increases lysosome escape includes but is not limited to an amino acid, a polyamino acid, an organic high-molecular polymer, a nucleic acid, a peptide, lipids, saccharides, and an inorganic substance with a proton sponge effect.

Furthermore, the surface of the nanoparticle or the microparticle is connected with a target head that actively targets the antigen-presenting cell, and the target head may be mannose, mannan, a CD19 antibody, a CD20 antibody, a BCMA antibody, a CD32 antibody, a CD11c antibody, a CD103 antibody, a CD44 antibody, etc.

Furthermore, the way in which the water-soluble antigen or the water-insoluble antigen is loaded on the surface of the nanoparticle or the microparticle includes at least one of adsorption, covalent linkage, charge interaction, hydrophobic interaction, one-step or multi-step solidification, mineralization, and encapsulation.

Furthermore, a particle size of the nanoparticle is 1 nm-1000 nm; a particle size of the microparticle is 1 μm-1000 μm; and the surface of the nanoparticle or the microparticle is electrically neutral, negatively charged, or positively charged.

Furthermore, the nanoparticle or the microparticle is prepared from an organic synthetic high-polymer material, a natural high-polymer material or an inorganic material, and may be prepared by an existing preparation method, including but not limited to a solvent evaporation method, a dialysis method, a microfluidic method, an extrusion method, and a hot melt method which are common.

Furthermore, the organic synthetic high-polymer material includes PLGA, PLA, PGA, PEG, PCL, poloxamer, PVA, PVP, PEI, PTMC, polyanhydride, PDON, PPDO, PMMA, polyamino acid, a synthetic peptide, etc; the natural high-polymer material includes lecithin, cholesterol, an alginate, albumin, collagen, gelatin, a cell membrane component, starch, saccharides, a peptide, etc.; and the inorganic material includes ferric oxide, ferroferric oxide, a carbonate, a phosphate, etc.

Furthermore, the nanoparticle or the microparticle may not be modified during a preparation process, or an appropriate modification technique may be adopted to increase the antigen-loading capacity of the nanoparticle or the microparticle. The modification technique includes but is not limited to biomineralization (e.g., silicification, calcification and magnesiation), gelation, crosslinking, chemical modification, addition of charged species, etc.

Furthermore, the form in which the antigen is loaded inside the nanoparticle or the microparticle may be any way in which the antigen may be loaded inside the nanoparticle or the microparticle, such as encapsulation.

Furthermore, the way in which the antigen is loaded on the surface of the nanoparticle or the microparticle includes but is not limited to adsorption, covalent linkage, charge interaction (e.g., addition of a positively charged species and addition of a negatively charged species), hydrophobic interaction, one-step or multi-step solidification, mineralization, encapsulation, etc.

Furthermore, the water-soluble antigen and/or the water-insoluble antigen loaded on the surface of the nanoparticle or the microparticle is one or more layers after being loaded, and when multiple layers of the water-soluble antigen and/or the water-insoluble antigen are loaded on the surface, there is a modifier between the layers.

Furthermore, a particle size of a particle used for activating or assisting isolation is nanoscale or microscale, which ensures that the particle is phagocytosed by the antigen-presenting cell, and in order to improve phagocytosis efficiency, the particle size should be within an appropriate range. The nanoparticle has a particle size of 1 nm-1000 nm, more preferably a particle size of 30 nm-1000 nm, and most preferably a particle size of 100 nm-600 nm; and the microparticle has a particle size of 1 μm-1000 μm, more preferably a particle size of 1 μm-100 μm, more preferably a particle size of 1 μm-10 μm, and most preferably a particle size of 1 μm-5 μm.

Furthermore, a shape of the nanoparticle or the microparticle loaded with the whole-cell antigen of the cancer cell includes but is not limited to spherical, ellipsoidal, barrel-shaped, polygonal, rod-shaped, sheet-like, linear, worm-shaped, square, triangular, butterfly-shaped, or disc-shaped.

Furthermore, when activating the cancer cell-specific T cell in vitro, the nanoparticle and/or the microparticle loaded with only the water-soluble antigen and the nanoparticle and/or the microparticle loaded with only the water-insoluble antigen may be used simultaneously, the nanoparticle and/or the microparticle loaded with only the water-soluble antigen may be used, the nanoparticle and/or the microparticle loaded with only the water-insoluble antigen may be used, or the nanoparticle and/or the microparticle simultaneously loaded with the water-soluble antigen and the water-insoluble antigen may be used.

Furthermore, the solubilizing agent is selected from at least one of urea, guanidine hydrochloride, a deoxycholate, a dodecyl sulfate (such as SDS), glycerol, a protein-degrading enzyme, albumin, lecithin, an inorganic salt (0.1-2000 mg/mL), Triton, Tween, an amino acid, a glycoside, and choline.

A second purpose of the present disclosure is to provide an application of the above cell system derived from a tumor-infiltrating lymphocyte in preparation of a medicament for treatment or prevention of cancer.

Furthermore, the medicament is administered multiple times before cancer occurrence, after cancer occurrence or after surgical excision of tumor tissue.

Furthermore, for the nanoparticle or the microparticle, at least one of the cancer cells or the tumor tissue used for preparing the antigen is of the same type as the target disease to be treated with the above medicament.

A third purpose of the present disclosure is to provide a method of activating a cancer cell-specific T cell in vitro, the method including steps of: co-incubating a nanoparticle and/or a microparticle loaded with the whole-cell antigen of the cancer cell and an antigen-presenting cell with a cancer cell-specific T cell or a cell mixture containing the cancer cell-specific T cell, where the whole-cell antigen of the cancer cell includes a water-soluble antigen and/or a water-insoluble antigen obtained by lysing the cancer cell and/or tumor tissue, and the water-insoluble antigen is loaded onto the nanoparticle or microparticle after being solubilized by a solubilizing agent or a solubilizing solution containing a solubilizing agent.

Furthermore, the cell mixture containing the cancer cell-specific T cell includes a tumor-infiltrating lymphocyte or a T cell derived from a tumor-infiltrating lymphocyte.

Furthermore, the solubilizing agent is selected from at least one of urea, guanidine hydrochloride, a deoxycholate, a dodecyl sulfate (such as SDS), glycerol, a protein-degrading enzyme, albumin, lecithin, an inorganic salt (0.1-2000 mg/mL), Triton, Tween, an amino acid, a glycoside, and choline.

Furthermore, the antigen-presenting cell includes one or more of a B cell, a dendritic cell and a macrophage.

Furthermore, a cytokine is added during co-incubating, and the cytokine includes but is not limited to an interleukin, an interferon, a colony-stimulating factor, and a tumor necrosis factor; and the interleukin includes but is not limited to interleukin 2 (IL-2), interleukin 7 (IL-7), interleukin 14 (IL-14), interleukin 4 (IL-4), interleukin 15 (IL-15), interleukin 21 (IL-21), interleukin 17 (IL-17), interleukin 12 (IL-12), interleukin 6 (IL-6), and interleukin 33 (IL-33).

A fourth purpose of the present disclosure is to provide a cancer cell-specific T cell activated in vitro by the above method.

In the prior art, due to the limitation of the way of activation, the number of types and clones of the cancer cell-specific T cell activated is very small. In the present disclosure, the whole-cell antigen of the cancer cell loaded on the nanoparticle or the microparticle for activating the cancer cell-specific T cell is derived from a cancer cell and/or tumor tissue. A water-insoluble antigen is loaded on the nanoparticle or the microparticle, so that the nanosystem or the microsystem contains more antigens, more preferably, a water-soluble antigen and a water-insoluble antigen are simultaneously loaded on the particle, so that the particle is loaded with all the antigens, and using the particle loaded with all cancer-related antigens to activate the cell can obtain a broader-spectrum and diverse cancer cell-specific T cell, which is highly specific and has a better effect in immunotherapy, thereby providing a more powerful candidate medicament for cell therapy.

Through the above solutions, the present disclosure has at least the following advantages.

The present disclosure provides a technique for isolating the cancer cell-specific T cell in the tumor-infiltrating lymphocyte after in vitro assisted activation using the nanoscale or microscale particle delivery system. The isolated cancer cell-specific T cell, which is broad-spectrum and highly specific, including all effector cancer cell-specific T cells in the tumor-infiltrating lymphocyte, is a T cell that may specifically recognize and kill a cancer cell. After the cancer cell-specific T cell is expanded, the obtained cell may be used to prevent and treat cancer.

The above description is merely an overview of the technical solutions of the present disclosure. In order to have a clearer understanding of the technical means of the present disclosure and to implement it according to the content of the specification, the preferred embodiments of the present disclosure are described below with detailed drawings.

### BRIEF DESCRIPTION OF THE DRAWINGS

In order to have a clearer understanding of the content of the present disclosure, the present disclosure will be described in further detail below with reference to specific embodiments of the present disclosure in conjunction with the accompanying drawings.

FIG. 1 is a schematic diagram of a preparation process and an application of a cell system according to the present disclosure, and includes FIG. 1A, FIG. 1B and FIG. 1C, where FIG. 1A is a schematic diagram of collecting and preparing nanoparticles or microparticles with water-soluble antigens and water-insoluble antigens, respectively; FIG. 1B is a schematic diagram of solubilizing whole-cell antigens of cancer cells and preparing nanoparticles or microparticles using a solubilizing solution containing a solubilizing agent; and FIG. 1C is a schematic diagram of activating cancer cell-specific T cells in tumor-infiltrating lymphocytes using the particles prepared in a or b, isolating and extracting the cancer cell-specific T cells using characteristics of activated T cells, then expanding the T cells, and using the cells to prevent or treat cancer.

FIGS. 2-20 respectively show experimental results of tumor growth rates and survival in mice when isolated and expanded cancer cell-specific T cells are used to prevent or treat cancer in Examples 1-19, where a shows experimental results of tumor growth rates when preventing or treating cancer (n≥8); b shows experimental results of mouse survival when preventing or treating cancer (n≥8), and each data point is mean±standard error of the mean (mean±SEM); c and d show results of analyzing a proportion of cancer cell-specific T cells to total tumor-infiltrating T cells activated by whole-cell antigens of cancer cells using flow cytometry; significant differences in tumor growth inhibition experiments in FIG. a were analyzed by the ANOVA method, and significant differences in FIG. b were analyzed by the Kaplan-Meier and log-rank test; *** indicates p<0.005 compared with PBS blank control group, with a significant difference; ** indicates p<0.01 compared with PBS blank control group, with a significant difference; * indicates p<0.05 compared with PBS blank control group, with a significant difference; ### indicates p<0.005 compared with T cell control group co-incubated with antigen-presenting cells only without any nanoparticles, with a significant difference; ## indicates p<0.01 compared with T cell control group co-incubated with antigen-presenting cells only without any nanoparticles/microparticles, with a significant difference; # indicates p<0.05 compared with T cell control group co-incubated with antigen-presenting cells only without any nanoparticles/microparticles, with a significant difference; &&& indicates that p<0.005 compared with T cell control group isolated with assistance of blank nanoparticles/microparticles+free lysate, with a significant difference; && indicates p<0.01 compared with T cell control group isolated with assistance of blank nanoparticles/microparticles+free lysate, with a significant difference; & indicates p<0.05 compared with T cell control group isolated with assistance of blank nanoparticles/microparticles+free lysate, with a significant difference; δδδ indicates p<0.005 compared with T cell group isolated with assistance of peptide nanoparticles/microparticles, with a significant difference; δδ indicates p<0.01 compared with T cell group isolated with assistance of peptide nanoparticles/microparticles, with a significant difference; Ω indicates p<0.05 compared with T cell group isolated with assistance of co-incubation of nanoparticles/microparticles and one kind of antigen-presenting cell, with a significant difference; π indicates p<0.05 compared with T cell group isolated with assistance of nanoparticles/microparticles not loaded with substances for lysosome escape, with a significant difference; ξ indicates p<0.05 compared with T cell group isolated with assistance of nanoparticles/microparticles loaded with only one kind of CpG+Poly(I:C) mixed adjuvant; μ indicates p<0.05 compared with T cell group isolated without cytokine addition during co-incubation in an assisted manner, with a significant difference; ρ indicates p<0.05 compared with T cell group isolated with assistance of nanoparticles/microparticles loaded with only one kind of adjuvant (two types of CpG), with a significant difference; ρρ indicates p<0.01 compared with T cell group isolated with assistance of nanoparticles/microparticles loaded with only one kind of adjuvant (two types of CpG), with a significant difference; ω indicates p<0.05 compared with cancer cell-specific CD8⁺ T cell group isolated with assistance of using only nanoparticles/microparticles, with a significant difference; θ indicates p<0.05 compared with T cell group isolated with assistance of nanoparticles/microparticles not loaded with adjuvants, with a significant difference; and 00 indicates p<0.01 compared with T cell group isolated with assistance of nanoparticles/microparticles not loaded with adjuvants, with a significant difference.

### DESCRIPTION OF THE EMBODIMENTS

The present disclosure will be further described below in conjunction with the accompanying drawings and specific embodiments in order to enable those skilled in the art to better understand and implement the present disclosure, but the embodiments provided are not intended to limit the present disclosure.

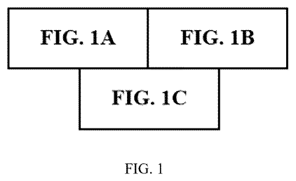

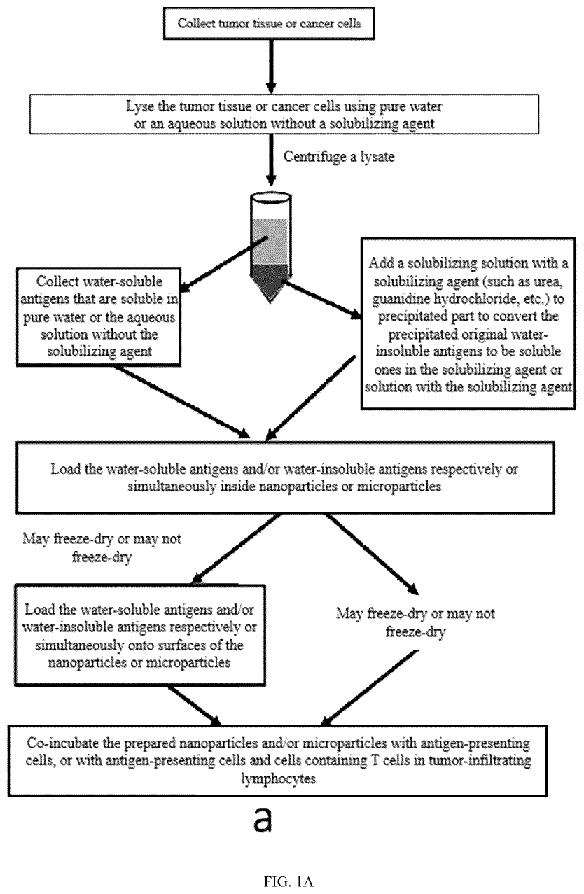

The T cell system for preventing or treating cancer according to the present disclosure includes specifically isolated and expanded cancer cell-specific T cells from tumor-infiltrating lymphocytes, and the cancer cell-specific T cells are activated by antigen-loaded nanoparticles and/or microparticles at the time of isolation, and then isolated using specific molecules highly expressed after activation. The cancer cell-specific T cells expanded after isolation may be derived from an autologous or allogeneic source. The nanoparticles and/or microparticles are loaded with whole-cell antigens of cancer cells or mixtures thereof. A T cell system for preventing or treating cancer is prepared, and the preparation process and application fields thereof are shown in FIG. 1.

When preparing nanoparticles or microparticles for assisting in isolating cancer cell-specific T cells, after lysing cells or tissue, water-soluble antigens and water-insoluble antigens may be collected respectively to prepare nanoparticle or microparticle systems respectively; or cells or tissue may be directly lysed by adopting a solubilizing agent solution containing a solubilizing agent, and whole-cell antigens of cancer cells may be solubilized to prepare nanoparticle or microparticle systems. The whole-cell antigens of cancer cells in the present disclosure may be treated before or (and) after lysis, including but not limited to inactivation or (and) denaturation, solidification, biomineralization, ionization, chemical modification, nuclease treatment, etc., before preparing nanoparticles or microparticles; and nanoparticles or microparticles may also be prepared directly before or (and) after cell lysis without any inactivation or (and) denaturation, solidification, biomineralization, ionization, chemical modification, and nuclease treatment. In some embodiments of the present disclosure, tumor tissue cells are subjected to inactivation or (and) denaturation treatment before lysis, and may be subjected to inactivation or (and) denaturation treatment after cell lysis in actual use, or may be subjected to inactivation or (and) denaturation treatment both before and after cell lysis; and in some embodiments of the present disclosure, the inactivation or (and) denaturation treatment methods before or (and) after cell lysis are ultraviolet irradiation and high-temperature heating, and the treatment methods including but not limited to radiation irradiation, high pressure, solidification, biomineralization, ionization, chemical modification, nuclease treatment, collagenase treatment, freeze-drying, etc. can also be adopted in the actual use process. Those skilled in the art may understand that in the actual application process, the skilled person may make appropriate adjustments according to specific situations.

When using nanoparticles or microparticles to activate cancer cell-specific T cells in vitro, the assistance of antigen-presenting cells is required, which may be derived from an autologous or allogeneic source, or from cell lines or stem cells. The antigen-presenting cells may be DC cells, B cells, macrophages, or any mixture of the three, or may be other cells having an antigen-presenting function.

After cancer cell-specific T cells are activated, the cancer cell-specific T cells specifically activated by the whole-cell antigens of cancer cells may be isolated and extracted by flow cytometry or magnetic bead sorting, or any other method that may extract and isolate such cells.

In some implementations, a specific preparation method for isolating and expanding cancer cell-specific T cells from tumor-infiltrating lymphocytes using nanoparticles or microparticles loaded with the whole-cell antigens of cancer cells is as follows:

Step 1: adding a first predetermined volume of an aqueous phase solution containing a first predetermined concentration to a second predetermined volume of an organic phase containing a second predetermined concentration of a raw material for particle preparation.

In some embodiments, the aqueous phase solution may contain the components in the cancer cell lysate and an immune-enhancing adjuvant; and each component in the cancer cell lysate is a water-soluble antigen or an original water-insoluble antigen solubilized in a solubilizing agent such as urea or guanidine hydrochloride at the time of preparation. The aqueous phase solution contains either a concentration of water-soluble antigen or a concentration of original water-insoluble antigen, i.e. a first predetermined concentration, which requires a protein/peptide concentration content greater than 1 ng/mL, to be capable of being loaded with sufficient whole-cell antigens of cancer cells to activate the relevant cells. The concentration of the immune-enhancing adjuvant in the initial aqueous phase is greater than 0.01 ng/mL.

In some embodiments, the aqueous phase solution contains the components in the tumor tissue lysate and an immune-enhancing adjuvant; and each component in the tumor tissue lysate is a water-soluble antigen or an original water-insoluble antigen solubilized in a solubilizing agent such as urea or guanidine hydrochloride at the time of preparation. The aqueous phase solution contains either a concentration of water-soluble antigen or a concentration of original water-insoluble antigen, i.e. a first predetermined concentration, which requires a protein and peptide concentration content greater than 0.01 ng/mL, to be capable of being loaded with sufficient whole-cell antigens of cancer cells to activate the relevant cells. The concentration of the immune-enhancing adjuvant in the initial aqueous phase is greater than 0.01 ng/mL.

In some embodiments, the raw material for preparing particles is PLGA, and the organic solvent selected is dichloromethane. Additionally, in some embodiments, the second predetermined concentration of the raw material for particle preparation ranges from 0.5 mg/mL to 5000 mg/mL, and preferably 100 mg/mL.

In the present disclosure, PLGA or modified PLGA is selected because the material is biodegradable and has been approved by the FDA for use as a drug dressing. Studies have shown that PLGA has a certain immunomodulatory function, so it is suitable as an excipient in the preparation of nanoparticles or microparticles. In practical applications, suitable materials may be selected according to actual situations.

In practice, the second predetermined volume of the organic phase is set according to its ratio to the first predetermined volume of the aqueous phase, and in the present disclosure, the ratio of the first predetermined volume of the aqueous phase to the second predetermined volume of the organic phase ranges from 1:1.1 to 1:5000, and preferably 1:10. In the specific implementation process, the first predetermined volume, the second predetermined volume, and the ratio of the first predetermined volume to the second predetermined volume may be adjusted as needed to adjust the size of the prepared nanoparticles or microparticles.

Preferably, when the aqueous phase solution is a lysate component solution, where the concentration of proteins and peptides is greater than 1 ng/mL, and preferably 1 mg/mL to 100 mg/mL; and when the aqueous phase solution is a lysate component/immune adjuvant solution, where the concentration of proteins and peptides is greater than 1 ng/mL, and preferably 1 mg/mL to 100 mg/mL, and the concentration of the immune adjuvant is greater than 0.01 ng/mL, and preferably 0.01 mg/mL to 20 mg/mL. In the organic phase solution, the solvent is DMSO, acetonitrile, ethanol, chloroform, methanol, DMF, isopropanol, dichloromethane, propanol, ethyl acetate, etc., and preferably dichloromethane; and the concentration of the organic phase is 0.5 mg/mL to 5000 mg/mL, and preferably 100 mg/mL.

Step 2, the mixed solution obtained in Step 1 is subjected to ultrasonic treatment for more than 2 seconds or stirring or homogenization treatment or microfluidic treatment for more than 1 minute. Preferably, when the stirring is mechanical stirring or magnetic stirring, the stirring speed is greater than 50 rpm and the stirring time is greater than 1 minute, for example, the stirring speed is 50 rpm to 1500 rpm and the stirring time is 0.1 hours to 24 hours; during the ultrasonic treatment, the ultrasonic power is greater than 5 W and the time is greater than 0.1 seconds, such as 2 seconds to 200 seconds; a high pressure/ultra-high pressure homogenizer or a high shear homogenizer is used for the homogenization treatment, the pressure is greater than 5 psi, such as 20 psi to 100 psi, when the high pressure/ultra-high pressure homogenizer is used, and the rotational speed is greater than 100 rpm, such as 1000 rpm to 5000 rpm, when the high shear homogenizer is used; and the microfluidic treatment is used with a flow rate greater than 0.01 mL/min, such as 0.1 mL/min to 100 mL/min. Ultrasonic, stirring, homogenization, or microfluidic treatment is used for nanocrystallization and/or micronization, the length of ultrasonic time, stirring speed, homogenization treatment pressure and time can control the size of the prepared microparticles and nanoparticles, and being too large and too small can cause changes in the particle size.

Step 3, adding the mixture obtained after the treatment of Step 2 to a third predetermined volume of an aqueous solution containing a third predetermined concentration of an emulsifier and performing ultrasonic treatment for more than 2 seconds or stirring for more than 1 minute or performing homogenization treatment or microfluidic treatment. In this step, the mixture obtained in Step 2 is added to the aqueous emulsifier solution to continue ultrasonic treatment or stirring for nanocrystallization or micronization. In the present disclosure, the ultrasonic time is greater than 0.1 seconds, such as 2 seconds to 200 seconds, the stirring speed is greater than 50 rpm, such as 50 rpm to 500 rpm, and the stirring time is greater than 1 minute, such as 60 seconds to 6000 seconds. Preferably, when the stirring is mechanical stirring or magnetic stirring, the stirring speed is greater than 50 rpm and the stirring time is greater than 1 minute, for example, the stirring speed is 50 rpm to 1500 rpm and the stirring time is 0.5 hours to 5 hours; during the ultrasonic treatment, the ultrasonic power is 50 W to 500 W and the time is greater than 0.1 seconds, such as 2 seconds to 200 seconds; a high pressure/ultra-high pressure homogenizer or a high shear homogenizer is used for the homogenization treatment, the pressure is greater than 20 psi, such as 20 psi to 100 psi, when the high pressure/ultra-high pressure homogenizer is used, and the rotational speed is greater than 1000 rpm, such as 1000 rpm to 5000 rpm, when the high shear homogenizer is used; and the microfluidic treatment is used with a flow rate greater than 0.01 mL/min, such as 0.1 mL/min to 100 mL/min. Ultrasonic, stirring, homogenization, or microfluidic treatment is used for nanocrystallization or micronization, the length of ultrasonic time, stirring speed, homogenization treatment pressure and time can control the size of the prepared microparticles or nanoparticles, and being too large and too small can cause changes in the particle size.

In some embodiments, the aqueous emulsifier solution is an aqueous polyvinyl alcohol (PVA) solution, the third predetermined volume is 5 mL, and the third predetermined concentration is 20 mg/mL. The third predetermined volume is adjusted according to its ratio to the second predetermined volume. In the present disclosure, the ratio of the second predetermined volume and the third predetermined volume is set to range from 1:1.1 to 1:1000, and preferably 2:5. In the specific implementation process, the ratio of the second predetermined volume to the third predetermined volume may be adjusted in order to control the size of the nanoparticles or microparticles. Similarly, the ultrasonic time or stirring time, and the volume and concentration of the aqueous emulsifier solution in this step are all determined to obtain nanoparticles or microparticles with appropriate sizes.

Step 4, adding the liquid obtained after the treatment in Step 3 to a fourth predetermined volume of an aqueous emulsifier solution having a fourth predetermined concentration, and stirring until a predetermined stirring condition is satisfied.

In this step, the aqueous emulsifier solution is a PVA solution or another solution.

The fourth predetermined concentration is 5 mg/mL, and the selection of the fourth predetermined concentration is based on obtaining nanoparticles or microparticles with appropriate sizes The selection of the fourth predetermined volume is determined according to the ratio of the third predetermined volume to the fourth predetermined volume. In the present disclosure, the ratio of the third predetermined volume to the fourth predetermined volume ranges from 1:1.5 to 1:2000, and preferably 1:10. In the specific implementation process, the ratio of the third predetermined volume to the fourth predetermined volume may be adjusted to control the size of nanoparticles or microparticles.

In the present disclosure, the predetermined stirring condition of this step is until the volatilization of the organic solvent is completed, that is, the volatilization of dichloromethane in Step 1 is completed.

Step 5, centrifuging the mixed solution that satisfies the predetermined stirring condition in Step 4 at a rotational speed greater than 100 RPM for more than 1 minute, then removing the supernatant, and resuspending the remaining precipitate in a fifth predetermined volume of a fifth predetermined concentration of an aqueous solution containing a freeze-drying protective agent or in a sixth predetermined volume of PBS (or normal saline).

In some implementations of the present disclosure, when the precipitate obtained in Step 5 is resuspended in the sixth predetermined volume of PBS (or normal saline), freeze-drying is not required, and subsequent experiments on adsorption of cancer cell lysates on the surface of nanoparticles or microparticles may be directly performed.

In some implementations of the present disclosure, when the precipitate obtained in Step 5 is resuspended in an aqueous solution containing a freeze-drying protective agent, freeze-drying is required, followed by subsequent experiments on the adsorption of cancer cell lysates on the surface of nanoparticles or microparticles.

In the present disclosure, the freeze-drying protective agent selected is trehalose.

In the present disclosure, the fifth predetermined concentration of the freeze-drying protective agent in this step is 4% in percentage by mass, which is set so as not to affect the freeze-drying effect during subsequent freeze-drying.

Step 6, freeze-drying the suspension containing the freeze-drying protective agent obtained in Step 5, and leaving the freeze-dried substance for later use.

Step 7, directly using the sixth predetermined volume of the nanoparticle-containing suspension resuspended in PBS (or normal saline) obtained in Step 5 or the freeze-dried substance containing nanoparticles or microparticles and the freeze-drying protective agent after freeze-drying obtained in Step 6 by resuspension using the sixth predetermined volume of PBS (or normal saline); or mixing the sample with a seventh predetermined volume of water-soluble antigens or solubilized original water-insoluble antigens for use.

In the present disclosure, the volume ratio of the sixth predetermined volume to the seventh predetermined volume is 1:10000 to 10000:1, preferably the volume ratio of 1:100 to 100:1, and most preferably the volume ratio of 1:30 to 30:1.

In some embodiments, the volume containing water-soluble antigens or solubilized original water-insoluble antigens in cancer cell lysates or tumor tissue lysates is 1 mL, when the volume of the resuspended nanoparticle suspension is 10 mL. In actual use, the volume and ratio of the two may be adjusted as needed.

Step 8, obtaining tumor tissue, cutting the tumor tissue into pieces, and then isolating and collecting live T cells from the tumor tissue. The tumor tissue may be derived from an autologous or allogeneic origin.

Step 9, mixing the nanoparticles and/or microparticles prepared in Step 7 with the T cells and antigen-presenting cells obtained in Step 8 and co-incubating for a certain period of time.

Step 10, isolating the T cells activated by whole-cell antigens of cancer cells by flow cytometry, magnetic bead sorting, etc.

Step 11, expanding the isolated T cells activated by whole-cell antigens of cancer cells in vitro.

Step 12, infusing the expanded cancer cell-specific T cells back into the body of patients to prevent or treat cancer.

In some other implementations, a specific preparation method for preparing antigen-loaded nanoparticles or microparticles is as follows:

Steps 1 to 4 are the same as above.

Step 5, centrifuging the mixed solution that satisfies the predetermined stirring condition in Step 4 at a rotational speed greater than 100 RPM for more than 1 minute, removing the supernatant, and resuspending the remaining precipitate in a fifth predetermined volume of a solution containing a fifth predetermined concentration of water-soluble and/or water-insoluble antigens in whole-cell antigens of cancer cells, or resuspending the remaining precipitate in a fifth predetermined volume of a solution containing a fifth predetermined concentration of water-soluble and/or water-insoluble antigens in whole-cell antigens of cancer cells and an adjuvant.

Step 6, centrifuging the mixed solution that satisfies the predetermined stirring condition in Step 5 at a rotational speed of more than 100 RPM for more than 1 minute, removing the supernatant, re-suspending the remaining precipitate in a sixth predetermined volume of a solidification treatment reagent or a mineralization treatment reagent for reaction for a certain period of time, then centrifuging and washing, and then adding a seventh predetermined volume of positively charged or negatively charged species for reaction for a certain period of time.

In some implementations of the present disclosure, after the precipitate obtained in Step 6 may be re-suspended in a seventh predetermined volume of charged species, freeze-drying is not required, and subsequent experiments on loading of cancer cell/tissue lysates onto the surface of nanoparticles or microparticles may be directly performed.

In some implementations of the present disclosure, the precipitate obtained in Step 6 is re-suspended in an aqueous solution containing a drying protective agent, then subjected to vacuum drying at room temperature or freeze vacuum drying, and then subsequent experiments on adsorption of cancer cell lysates on the surface of nanoparticles or microparticles are performed after drying.

In the present disclosure, the freeze-drying protective agent selected is trehalose or a mixed solution of mannitol and sucrose. In the present disclosure, the concentration of the drying protective agent in this step is 4% in percentage by mass, which is set so as not to affect the drying effect during subsequent drying.

Step 7, drying the suspension containing the drying protective agent obtained in Step 6, and leaving the dried substance for later use.

Step 8, directly using the eighth predetermined volume of the nanoparticle-containing suspension resuspended in PBS (or normal saline) obtained in Step 6 or the dried substance containing nanoparticles or microparticles and the drying protective agent after drying obtained in Step 7 by resuspension using the eighth predetermined volume of PBS (or normal saline); or mixing with a ninth predetermined volume of water-soluble antigens or water-insoluble antigens for use.

In the present disclosure, the modification and antigen-loading steps of Steps 5-8 may be repeated a plurality of times to increase the antigen-loading capacity. Moreover, when adding positively charged or negatively charged species, species with the same charges may be added a plurality of times or species with different charges may be added alternately.

In some embodiments, the volume containing water-soluble antigens or original water-insoluble antigens in cancer cell lysates or tumor tissue lysates is 0.1 mL to 100 mL, when the volume of the resuspended nanoparticle suspension is 10 mL. In actual use, the volume and ratio of the two may be adjusted as needed.

Step 9, obtaining tumor tissue, cutting the tumor tissue into pieces, and then isolating and collecting live T cells from the tumor tissue. The tumor tissue may be derived from an autologous or allogeneic origin.

Step 10, mixing the nanoparticles and/or microparticles prepared in Step 8 with the T cells and antigen-presenting cells obtained in Step 9 and co-incubating for a certain period of time.

Step 11, isolating the T cells activated by whole-cell antigens of cancer cells by flow cytometry, magnetic bead sorting, etc.

Step 12, expanding the isolated T cells activated by whole-cell antigens of cancer cells in vitro.

Step 13, infusing the expanded cancer cell-specific T cells back into the body of patients to prevent or treat cancer.

### Example 1 Isolated and Expanded Cancer Cell-Specific T Cells for Melanoma Prevention

This example used mouse melanoma as a cancer model to illustrate how nanoparticle-assisted isolation and expansion of cancer cell-specific T cells in tumor-infiltrating lymphocytes was used for melanoma prevention. In this example, B16F10 melanoma tumor tissue was lysed to prepare water-soluble antigens and water-insoluble antigens of the tumor tissue, then a nanoparticle system loaded with water-soluble antigens and water-insoluble antigens of the tumor tissue was prepared by a solvent evaporation method using organic polymer material PLGA as a nanoparticle skeleton material and polyinosinic-polycytidylic acid (poly(I:C)) as an immune adjuvant, and then the nanoparticles were used to assist in isolating cancer cell-specific T cells in tumor-infiltrating lymphocytes, and the isolated cancer cell-specific T cells were expanded and injected into the body to prevent melanoma.

### (1) Lysis and Component Collection of Tumor Tissue

Each C57BL/6 mouse was subcutaneously inoculated with 1.5×10⁵ B16F10 cells on the back, and the mice were sacrificed and the tumor tissue removed when the tumors each grew to a volume of approximately 1000 mm³. The tumor tissue was cut into pieces and ground, the sample was filtered through a cell strainer and an appropriate amount of ultrapure water was added, and freezing-thawing cycle was repeated 5 times, accompanied by ultrasound to destroy and lyse the cells. After the cells were lysed, the lysate was centrifuged at a rotational speed of 5000 g for 5 minutes, and the supernatant was taken to obtain water-soluble antigens that were soluble in pure water; and adding 8 M urea to the obtained precipitated fraction to solubilize the precipitated fraction can convert the water-insoluble antigens that were insoluble in pure water into soluble ones in an 8 M aqueous urea solution. The water-soluble antigens and the water-insoluble antigens were mixed according to a mass ratio of 1:1, which was the source of antigen raw materials for preparing the nanoparticle system.

### (2) Preparation of a Nanoparticle System

In this example, the nanovaccines and the blank nanoparticles as controls were prepared by a double-emulsion method in the solvent evaporation method. The molecular weight of the nanoparticle preparation material PLGA adopted was 24 KDa to 38 KDa, the immune adjuvant applied was poly(I:C), and the poly(I:C) was only distributed inside the nanoparticles. The preparation method was as previously described. In the preparation process, the cell components and adjuvant were first loaded inside the nanoparticles by the double-emulsion method, and after the cell lysis components were loaded inside, 100 mg nanoparticles were centrifuged at 10000 g for 20 minutes and freeze-dried for 48 h after resuspension using 10 mL of ultrapure water containing 4% trehalose. The average particle size of the nanoparticles was about 280 nm, and the surface potential of the nanoparticles was about −3 mV; and approximately 100 μg of the protein or peptide components were loaded per 1 mg of PLGA nanoparticles, and 0.02 mg of the poly(I:C) immune adjuvant was used per 1 mg of PLGA nanoparticles. The particle size of the blank nanoparticles was about 260 nm. When preparing the blank nanoparticles, pure water containing an equivalent amount of poly(I:C) or 8 M urea was utilized to replace the corresponding water-soluble antigens and water-insoluble antigens.

### (3) Isolation and Expansion of Cancer Cell-Specific T Cells

Each C57BL/6 mouse was subcutaneously inoculated with 0.5×10⁵ B16F10 cells on the back, and the mice were sacrificed and the tumor tissue and splenocytes were harvested when the tumors each grew to a volume of approximately 1000 mm³. The mouse tumor tissue was digested with collagenase for 15 minutes after cutting into small pieces, then a single-cell suspension was prepared through a cell strainer, and CD3⁺ T cells were isolated from live cells (dead cells were labeled with live-dead cell dye to remove dead cells) in the tumor tissue single-cell suspension using flow cytometry after centrifugation and washing with PBS. At the same time, a splenocyte single-cell suspension was prepared after the mouse spleen was passed through a cell strainer and red blood cells were lysed, and CD19⁺ B cells were sorted from live cells in the splenocyte single-cell suspension using flow cytometry (dead cells were labeled with live-dead cell dye to remove dead cells). The nanoparticles loaded with whole-cell antigens of cancer cells derived from tumor tissue (50 μg), B cells (2 million cells) and T cells from tumor-infiltrating lymphocytes (0.5 million cells) were co-incubated in 3 mL of RPMI 1649 complete medium for 96 hours (37° C., 5% CO₂); or blank nanoparticles (50 μg)+ equal quantity of a free lysate, B cells (2 million cells) and T cells from tumor-infiltrating lymphocytes (0.5 million cells) were co-incubated in 3 mL of RPMI 1649 complete medium for 96 hours (37° C., 5% CO₂); or B cells (2 million cells) and T cells from tumor-infiltrating lymphocytes (0.5 million cells) were co-incubated in 3 mL of RPMI 1649 complete medium for 96 hours (37° C., 5% CO₂). The incubated cells were then sorted by flow cytometry for CD3⁺ CD8⁺ CD69⁺ T cells, i.e. cancer cell-specific CD8⁺ T cells. The cancer cell-specific T cells obtained by the above sorting were co-incubated with IL-2 (2000 U/mL), IL-12 (200 U/mL), IL-15 (200 U/mL), and αCD-3 antibodies (10 ng/mL) for 10 days (medium change every two days) to expand the sorted cancer cell-specific T cells.

### (4) Cancer Cell-Specific T Cells for Cancer Prevention

Female C57BL/6 mice aged 6-8 weeks were selected as model mice to prepare melanoma tumor-bearing mice. One day before the adoptive transfer of cells, the recipient mice were intraperitoneally injected with a 100 mg/kg dose of cyclophosphamide to eliminate immune cells in the recipient mice. Then, the 4 million cancer cell-specific T cells prepared in step (3) were intravenously injected into the recipient mice. Each recipient mouse was subcutaneously inoculated with 1.5×10⁵ B16F10 cells on the lower right side of the back the next day. The tumor growth rate of the mice and the survival of the mice were monitored. In the experiment, the size of tumor volume in the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (5) Experimental Results

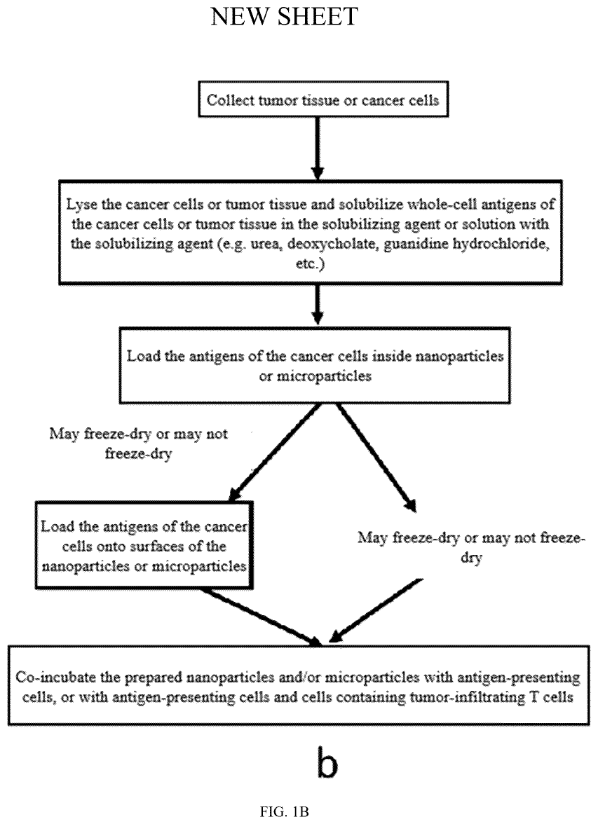

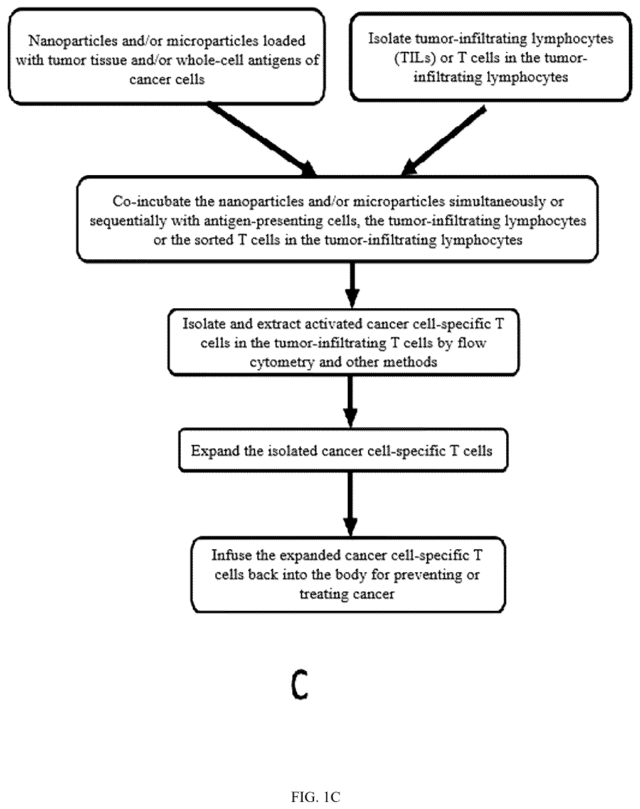

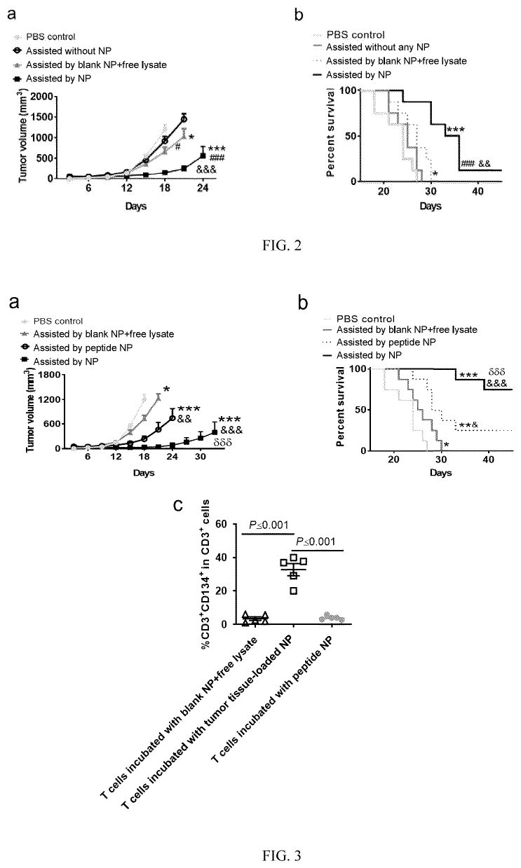

As shown in FIG. 2, the PBS control group and the mice treated with cancer cell-specific T cells without nanoparticle-assisted isolation and expansion had a fast tumor growth rate and a short survival time. The tumor growth rate of the mice treated with cancer cell-specific T cells isolated and expanded with assistance of blank nanoparticles+free lysate slowed down. The mice treated with cancer cell-specific T cells isolated and expanded with assistance of nanoparticles loaded with whole-cell antigens of cancer cells had the slowest tumor growth rate and the longest survival time. In summary, the cancer cell-specific T cells of the present disclosure had a favorable preventive effect on melanoma.

### Example 2 Isolated and Expanded Cancer Cell-Specific T Cells for Melanoma Prevention

This example used mouse melanoma as a cancer model to illustrate how nanoparticle-assisted isolation and expansion of cancer cell-specific T cells were used for melanoma prevention. In this example, B16F10 melanoma tumor tissue was lysed to prepare water-soluble antigens and water-insoluble antigens of the tumor tissue, then a nanoparticle system loaded with water-soluble antigens and water-insoluble antigens of the tumor tissue was prepared by a solvent evaporation method using PLGA as a nanoparticle skeleton material and poly(I:C) and CpG1018 as immune adjuvants, and then the nanoparticles were used to assist in isolating cancer cell-specific T cells in tumor-infiltrating lymphocytes, and the isolated cancer cell-specific T cells were expanded and injected into the body to prevent melanoma.

### (1) Lysis and Component Collection of Tumor Tissue

Each C57BL/6 mouse was subcutaneously inoculated with 1.5×10⁵ B16F10 cells on the back, and the mice were sacrificed and the tumor tissue removed when the tumors each grew to a volume of approximately 1000 mm³. The tumor tissue was cut into pieces and ground, an appropriate amount of pure water was added through a cell strainer, and freezing-hawing cycle was repeated 5 times, accompanied by ultrasound to destroy and lyse the cells. After the cells were lysed, the lysate was centrifuged at a rotational speed of 5000 g for 5 minutes, and the supernatant was taken to obtain water-soluble antigens that were soluble in pure water; and adding 8 M urea to the obtained precipitated fraction to solubilize the precipitated fraction can convert the water-insoluble antigens that were insoluble in pure water into soluble ones in an 8 M aqueous urea solution. The above was the source of antigen raw materials for preparing the nanoparticle system.

### (2) Preparation of a Nanoparticle System

In this example, the nanovaccines and the blank nanoparticles as controls were prepared by the solvent evaporation method. At the time of preparation, the nanovaccines loaded with the water-soluble antigens in the whole-cell antigens of cancer cells and the nanoparticles loaded with the water-insoluble antigens in the whole-cell antigens of cancer cells were respectively prepared and then used together when used. The molecular weight of the nanoparticle preparation material PLGA adopted was 7 Da to 17 KDa, and the immune adjuvants adopted were poly(I:C) and CpG1018, and the adjuvants were encapsulated inside the nanoparticles. The preparation method was as previously described. In the preparation process, the antigens and adjuvants were first loaded inside the nanoparticles by the double-emulsion method, and after the antigens (lysis components) were loaded inside, 100 mg nanoparticles were centrifuged at 10000 g for 20 minutes and freeze-dried for 48 h after resuspension using 10 mL of ultrapure water containing 4% trehalose. The average particle size of the nanoparticles was about 280 nm; approximately 100 μg of the protein and peptide components were loaded per 1 mg of PLGA nanoparticles, and 0.02 mg each of poly(I:C) and CpG1018 immune adjuvants were used per 1 mg of PLGA nanoparticles. In this example, nanoparticles loaded with four peptide neoantigens B16-M20 (Tubb3, FRRKAFLHWYTGEAMDEMEFTEAESNM)(SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3) and TRP2:180-188(SVYDFFVWL) (SEQ ID NO: 4) in equivalent mass were used as control nanoparticles. The control nanoparticles had a particle size of about 260 nm, loaded with 100 μg of the peptide components and loaded with an equivalent quantity of the adjuvants. The particle size of the blank nanoparticles was about 250 nm, and only an equivalent quantity of the immune adjuvants was loaded but no antigen component was loaded.

### (3) Isolation and Expansion of Cancer Cell-Specific T Cells

On day 0, 5×10⁵ B16F10 cells were subcutaneously inoculated on the back of each C57BL/6 mouse, and the mice were subcutaneously injected with 100 μL of 1 mg PLGA nanoparticles containing the water-soluble antigens and 100 μL of 1 mg PLGA nanoparticles containing the water-insoluble antigens on days 7, 14, and 28, respectively. The mice were sacrificed on day 32, and the spleen and tumor tissue of the mice were collected. A single-cell suspension was prepared through a cell strainer after cutting the mouse tumor tissue into small pieces, and CD3⁺ T cells in live cells (dead cells were labeled with live-dead cell dye to remove dead cells) were isolated from the tumor tissue single-cell suspension using flow cytometry after centrifugation and washing with PBS. At the same time, a splenocyte single-cell suspension was prepared after the mouse spleen was passed through a cell strainer and red blood cells were lysed, and CD19⁺ B cells in live cells were sorted from the splenocyte single-cell suspension using flow cytometry (dead cells were labeled with live-dead cell dye to remove dead cells). The nanoparticles (100 μg) loaded with the whole-cell antigens of cancer cells derived from tumor tissue or peptide nanoparticles (100 μg) or blank nanoparticles (100 μg)+free lysate were co-incubated with B cells (2 million cells), DC2.4 cells (2 million cells) and T cells from tumor-infiltrating lymphocytes (0.4 million cells) in 5 mL RPMI 1640 complete medium for 48 hours (37° C., 5% CO₂), and then the incubated CD3⁺ CD134⁺ T cells, i.e., cancer cell-specific T cells activated by the whole-cell antigens of cancer cells, were sorted by flow cytometry. At the same time, flow cytometry was used to analyze the proportion of CD3⁺ CD134⁺ T cells to CD3⁺ T cells after co-incubation of different nanoparticles with T cells and antigen-presenting cells. The whole-cell antigens of cancer cells loaded by nanoparticles can be degraded into antigen epitopes after being phagocytosed by antigen-presenting cells (B cells or DC cells) and presented to the surface of antigen-presenting cells. The specific T cells that can recognize the whole-cell antigens of cancer cells can recognize the whole-cell antigen epitopes of cancer cells and then be activated and express specific surface markers. By analyzing the proportion of T cells that highly expressed specific surface markers by flow cytometry, the number of cancer cell-specific T cells that were activated and can be sorted out that can recognize the cancer cells and had killing efficiency can be determined.

The cancer cell-specific T cells obtained by the above sorting were co-incubated with IL-2 (2000 U/mL) and αCD-3 antibodies (20 ng/mL) for 14 days (medium change every two days) to expand the sorted cancer cell-specific T cells.

### (4) Cancer Cell-Specific T Cells for Cancer Prevention

Female C57BL/6 mice aged 6-8 weeks were selected as model mice to prepare melanoma tumor-bearing mice. One day before the adoptive transfer of cells, the recipient mice were intraperitoneally injected with a 100 mg/kg dose of cyclophosphamide to eliminate immune cells in the recipient mice. Then, 1 million cancer cell-specific T cells prepared in step (3) were intravenously injected into the recipient mice. Each recipient mouse was subcutaneously inoculated with 1.5×10⁵ B16F10 cells on the lower right side of the back the next day. The tumor growth rate of the mice and the survival of the mice were monitored. In the experiment, the size of tumor volume in the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (5) Experimental Results

As shown in FIG. 3, the tumor growth rate of the mice in the PBS control group and blank nanoparticle control group was very fast, and the survival time of the mice was very short. Compared with the above two groups of control groups, the tumor growth rate of the recipient mice receiving cancer cell-specific T cells isolated and expanded with assistance of nanoparticles was significantly slower, and the tumors of some mice disappeared and recovered. Moreover, the cancer cell-specific T cells isolated and expanded with assistance of nanoparticles loaded with the whole-cell antigens of cancer cells were more effective in preventing cancer than the cancer cell-specific T cells isolated and expanded with assistance of nanoparticles loaded with four antigen peptides. This indicated that the types of cancer cell-specific T cells that can be isolated with assistance of four neoantigen peptide nanoparticles were limited, so the number of T cell clones contained in the expanded T cell system was very small, and the cancer cells that can be recognized and killed were fewer. The nanoparticles loaded with the whole-cell antigens of cancer cells can help isolate wider-spectrum cancer cell-specific T cells, so the number of T cell clones that can be obtained after expansion was wider-spectrum, and the more cancer cells that can be identified and killed, the better the effect of treating or preventing cancer.

### Example 3 Sorted and Expanded Cancer Cell-Specific T Cells for Melanoma Treatment

This example used mouse melanoma as a cancer model to illustrate how nanoparticle-assisted isolation and expansion of cancer cell-specific T cells in tumor tissue-infiltrating lymphocytes was used for melanoma treatment. In this example, B16F10 melanoma tumor tissue and cancer cells were first lysed to prepare a water-soluble antigen mixture (mass ratio 1:1) and a water-insoluble antigen mixture (mass ratio 1:1) of the tumor tissue and cancer cells, and the water-soluble antigen mixture and the water-insoluble antigen mixture were mixed at a mass ratio of 1:1. Then, PLGA was used as a nanoparticle skeleton material, Poly(I:C) and CpG2006 were used as adjuvants to prepare nanoparticles loaded with lysate components, then the nanoparticles were co-incubated with T cells and antigen-presenting cells for a certain period of time in vitro to activate cancer cell-specific T cells and isolate cancer cell-specific T cells by using surface markers highly expressed after T cells activation, and the activated cancer cell-specific T cells were used to treat melanoma after expansion.

### (1) Lysis and Component Collection of Tumor Tissue and Cancer Cells

When collecting tumor tissue, 1.5×10⁵ B16F10 cells were subcutaneously inoculated on the back of each C57BL/6 mouse. When the tumor grew to a volume of about 1000 mm³, the mice were sacrificed and the tumor tissue was removed. The tumor tissue was cut into pieces and ground, the sample was filtered through the cell strainer and an appropriate amount of pure water was added, and freezing-thawing cycle was repeated 5 times, and ultrasound can be used to destroy and lyse the obtained samples. When collecting the cultured B16F10 cancer cell line, the culture medium was removed by centrifugation, then the cancer cells were washed twice with PBS, and the cancer cells were collected by centrifugation. The cancer cells were resuspended in ultrapure water and repeatedly frozen and thawed 3 times, accompanied by ultrasound to destroy and lyse the cells. After the tumor tissue or cancer cell was lysed, the lysate was centrifuged at a rotational speed of 5000 g for 5 minutes, and the supernatant was taken to obtain water-soluble antigens that were soluble in pure water; and adding 8 M urea to the obtained precipitated fraction to solubilize the precipitated fraction can convert the water-insoluble antigens that were insoluble in pure water into soluble ones in an 8 M aqueous urea solution. The water-soluble antigens of the tumor tissue and the water-soluble antigens of the cancer cells were mixed at a mass ratio of 1:1; and the water-insoluble antigens of the tumor tissue and the water-insoluble antigens of the cancer cells were mixed at a mass ratio of 1:1. The water-soluble antigen mixture and the water-insoluble antigen mixture were mixed at a mass ratio of 1:1, which was the source of antigen raw materials for preparing the nanoparticles.

### (2) Preparation of Nanoparticles

The nanoparticles in this example were prepared by a double-emulsion method. The molecular weight of the nanoparticle preparation material PLGA adopted was 7 KDa to 17 KDa, and the immune adjuvants adopted were poly(I:C) and CpG2006, and the adjuvants were encapsulated inside the nanoparticles. The preparation method was as previously described. In the preparation process, the lysate components and adjuvants were first loaded inside the nanoparticles by the double-emulsion method. After the lysate components were loaded inside, 100 mg nanoparticles were centrifuged at 10000 g for 20 minutes, and resuspended with 10 mL of ultrapure water containing 4% trehalose and then freeze-dried for 48 h; and before use, the nanoparticles were resuspended in 9 mL of PBS and then 1 mL of the lysate components (protein concentration of 80 mg/mL) was added for reaction at room temperature for 10 min to obtain a nanoparticle system loaded with the lysate both inside and outside. The average particle size of the nanoparticles was about 280 nm, and the surface potential of the nanoparticles was about −5 mV; and approximately 130 μg of the protein or peptide components were loaded per 1 mg of PLGA nanoparticles, and 0.02 mg each of poly(I:C) and CpG2006 immune adjuvants were loaded per 1 mg of PLGA nanoparticles. The particle size of the blank nanoparticles was about 260 n, and the blank nanoparticles were prepared with an equal quantity of the adjuvants.

### (3) Isolation and Expansion of Cancer Cell-Specific T Cells

Each C57BL/6 mouse was subcutaneously inoculated with 5×10⁵ B16F10 cells on the back on day 0, and the mice were subcutaneously injected with 0.5 mg PLGA nanoparticles on days 10, 17 and 24, respectively. The mice were sacrificed on day 31, and the tumor tissue and spleen of the mice were harvested. A single-cell suspension of mouse tumor tissue was prepared, and CD45⁺ CD3⁺ T cells were sorted from live cells in tumor-infiltrating lymphocytes (dead cells were labeled with live-dead cell dye to remove dead cells) using magnetic bead sorting. A single-cell suspension of splenocytes was prepared, and CD19⁺ B cells were isolated from live cells (dead cells were labeled with live-dead cell dye to remove dead cells) in splenocytes using magnetic bead sorting. The isolated B cells (3 million cells), DC2.4 cells (2 million cells) and T cells (0.4 million cells) were incubated with nanoparticles (80 μg) or blank nanoparticles (80 μg)+free lysate in 40 mL high-glucose DMEM complete medium for 72 hours (37° C., 5% CO₂), and then the incubated CD3⁺ CD69⁺ T cells, i.e., the cancer cell-specific T cells specifically activated by the whole-cell antigens of cancer cells, were sorted by flow cytometry. At the same time, flow cytometry was used to analyze the proportion of CD3⁺ CD69⁺ T cells in tumor tissue-infiltrating T cells without nanoparticle-assisted sorting and tumor tissue-infiltrating T cells after co-incubation with nanoparticles and antigen-presenting cells. At the same time, tumor tissue-infiltrating T cells without nanoparticle-assisted sorting or tumor tissue-infiltrating T cells co-incubated with nanoparticles and antigen-presenting cells were incubated with B cells (3 million cells), DC2.4 cells (2 million cells) and nanoparticles loaded with whole-cell antigens of cancer cells (100 μg) in 3 mL DMEM high-glucose complete medium for 48 hours, then the incubated cells were collected and labeled with IFN-γ antibodies with fluorescent probes, and then the proportion of IFN-γ⁺ T cells in T cells was analyzed by flow cytometry. The whole-cell antigens of cancer cells loaded by nanoparticles can be degraded into antigen epitopes after being phagocytosed by antigen-presenting cells and presented to the surface of antigen-presenting cells. Specific T cells that can recognize the whole-cell antigens of cancer cells can recognize the whole-cell antigen epitopes of cancer cells and then be activated and secrete killer cytokines. IFN-γ was the most important cytokine secreted by antigen-specific T cells that were activated after recognizing antigens. However, because IFN-γ was a secretory cytokine, it needed to be stained with antibodies after cell fixation and membrane rupture (the cells were dead cells after analysis). CD3⁺ IFN-γ+ T cells analyzed using flow cytometry were cancer cell-specific T cells that can recognize and kill cancer cells.

The sorted cancer cell-specific T cells were incubated with IL-2 (2000 U/mL) in DMEM high glucose complete medium for 7 days (37° C., 5% CO₂, medium change every two days) to expand the sorted cancer cell-specific T cells.

### (4) Cancer Cell-Specific T Cells for Cancer Treatment

Female C57BL/6 aged 6-8 weeks were selected as model mice to prepare melanoma-bearing mice. Each mouse was subcutaneously inoculated with 1.5×10⁵ B16F10 cells on the lower right side of the back on day 0. Two million cancer cell-specific T cells were intravenously injected on day 4, day 7, day 10, day 15, day 20, and day 25 respectively after melanoma inoculation. In the experiment, the size of tumor volume in the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (5) Experimental Results

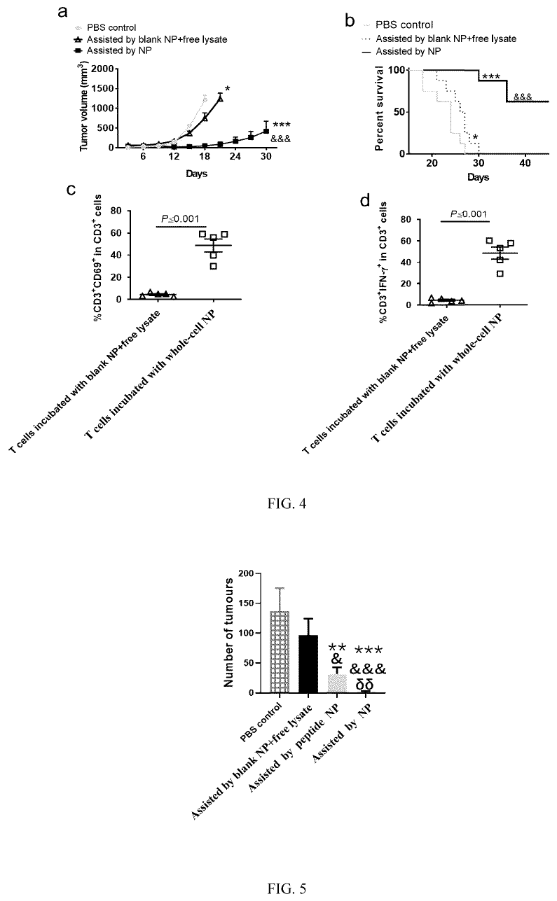

As shown in FIGS. 4a and 4b, the tumor growth rate of the mice in the PBS control group and blank nanoparticle control group was fast, while the tumor growth rate of the mice treated with cancer cell-specific T cells isolated and expanded with assistance of nanoparticles was significantly slower, and the tumors of some mice disappeared and recovered. In summary, the cell system of the present disclosure had an excellent therapeutic effect on cancer.

As shown in FIGS. 4c and 4d, after the nanoparticles were co-incubated with antigen-presenting cells and tumor-infiltrating T cells, the proportion of CD3⁺ IFN-γ⁺ T cells or CD3⁺ CD69⁺ T cells to CD3⁺ T cells was significantly higher than that of the blank nanoparticles+free lysate group. Moreover, the proportions of CD3⁺ IFN-γ⁺ T cells and CD3⁺ CD69⁺ T cells in CD3⁺ T cells were basically the same. Therefore, the isolation method of the present disclosure can effectively enrich cancer cell-specific T cells having the ability to recognize cancer cells and kill cancer cells in tumor tissue.

### Example 4 Sorted and Expanded Cancer Cell-Specific T Cells for Prevention of Melanoma Lung Metastasis

In this example, mouse melanoma lung models were used to illustrate how nanoparticles were used to assist in isolating cancer cell-specific T cells, and the expanded cells were used to prevent cancer metastasis. In this example, first, B16F10 melanoma tumor tissue was lysed to prepare water-soluble antigens and water-insoluble antigens of the tumor tissue; and then, nanoparticle systems loaded with the water-soluble antigens and water-insoluble antigens of the tumor tissue were prepared. In this example, silicification and addition of charged species were performed to increase the antigen-loading capacity, and only one round of mineralization treatment was performed. In this example, nanoparticles were used to assist in isolating cancer cell-specific T cells in tumor-infiltrating lymphocytes, and then the cancer cell-specific T cells were expanded in vitro and used by injection.

### (1) Lysis and Component Collection of Tumor Tissue

Each C57BL/6 mouse was subcutaneously inoculated with 1.5×10⁵ B16F10 cells on the back, and the mice were sacrificed and the tumor tissue removed when the tumors each grew to a volume of approximately 1000 mm³. The tumor tissue was cut into pieces and ground, added with collagenase and incubated in RPMI 1640 medium for 30 min, then an appropriate amount of pure water was added through a cell strainer, and freezing-thawing cycle was repeated 5 times, accompanied by ultrasound to destroy and lyse the cells. After the cells were lysed, the lysate was centrifuged at a rotational speed of 5000 g for 5 minutes, and the supernatant was taken to obtain water-soluble antigens that were soluble in pure water; and adding 8 M urea to the obtained precipitated fraction to solubilize the precipitated fraction can convert the water-insoluble antigens that were insoluble in pure water into soluble ones in an 8 M aqueous urea solution, and the water-soluble antigens and the water-insoluble antigens were mixed at a mass ratio of 2:1, which were the source of antigen raw materials for preparing the particles.

### (2) Preparation of Nanoparticles

In this example, the nanoparticles and the blank nanoparticles as controls were prepared by a solvent evaporation method, and appropriate modification and improvement were performed. In the preparation process of the nanoparticles, two modification methods, low-temperature silicification technology and addition of charged species, were adopted to increase the antigen-loading capacity. At the time of preparation, the nanoparticles loaded with the water-soluble antigens in the whole-cell antigens of cancer cells and the nanoparticles loaded with the water-insoluble antigens in the whole-cell antigens of cancer cells were respectively prepared and then used together when used. The molecular weight of the nanoparticle preparation material PLGA applied was 24 KDa to 38 KDa, and the immune adjuvant applied was poly(I:C). The preparation method was as previously described. In the preparation process, the antigens and adjuvant were first loaded inside the nanoparticles by the double-emulsion method, after the antigens (lysis components) were loaded inside, 100 mg of the nanoparticles were centrifuged at 10000 g for 20 minutes, then the nanoparticles were resuspended using 7 mL of PBS and mixed with 3 mL of a PBS solution containing the cell lysate (60 mg/mL), and then centrifuged at 10000 g for 20 minutes. Then the nanoparticles were resuspended with 10 mL of a silicate solution (containing 150 mM NaCl, 80 mM tetramethyl orthosilicate and 1.0 mM HCl, pH 3.0) and fixed at room temperature for 10 min, then fix at −80° C. for 24 h, centrifuged and washed with ultrapure water and then resuspended with 3 mL of PBS containing protamine (5 mg/mL) and polylysine (10 mg/mL) for reaction for 10 min, then washed by centrifugation at 10000 g for 20 min, resuspended with 10 mL of a PBS solution containing the cell lysate (50 mg/mL) for 10 min, and then freeze-dried for 48 h after centrifugation at 10000 g for 20 min and resuspension with 10 mL of ultrapure water containing 4% trehalose; and before use, the particles were resuspended in 7 mL of PBS, and then added with 3 mL of adjuvant-containing cancer tissue lysate components (protein concentration of 50 mg/mL) for reaction at room temperature for 10 min to obtain a frozen silicified and modified nanoparticle system with cationic substances loaded with the lysate both inside and outside. The average particle size of the nanoparticles was about 350 nm, and the surface potential of the nanoparticles was about −3 mV; and approximately 300 μg of the protein or peptide components were loaded per 1 mg of PLGA nanoparticles, and a total of about 0.02 mg of the poly(I:C) immune adjuvant was used inside and outside per 1 mg of PLGA nanoparticles, with half inside and half outside.

The loaded whole-cell antigens of cancer cells were replaced with four melanoma antigen peptides of an equivalent amount of mass in the control nanoparticles, and the rest was the same as the nanoparticles loaded with the whole-cell antigens of cancer cells. The control nanoparticles used 0.02 mg of poly(I:C) per 1 mg of PLGA nanoparticles, the average particle size was about 350 nm, and the nanoparticle surface potential was about −3 mV. The four loaded peptide neoantigens were B16-M20 (Tubb3, FRRKAFLHWYTGEAMDEMEFTEAESNM) (SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3), and TRP2:180-188 (SVYDFFVWL) (SEQ ID NO: 4).

The particle size of the blank nanoparticles was about 300 nm. When preparing the blank nanoparticles, pure water containing an equivalent amount of poly(I:C) or 8 M urea was applied to replace the corresponding water-soluble antigens and water-insoluble antigens.

### (3) Preparation of Dendritic Cells

This example illustrated how to prepare bone marrow-derived dendritic cells (BMDCs) by taking the preparation of dendritic cells from mouse bone marrow cells as an example. First, a 6-8-week-old C57 mouse was sacrificed by cervical dislocation. The tibia and femur of the hind leg were surgically removed and put into PBS, and the muscle tissue around the bone was removed cleanly with scissors and tweezers. Both ends of the bone were cut off with scissors, then a PBS solution was extracted with a syringe, needles were inserted from both ends of the bone into the bone marrow cavity, and the bone marrow was repeatedly rinsed into a culture dish. The bone marrow solution was collected, centrifuged at 400 g for 3 min, and then added with 1 mL of a red blood cell lysate to lyse red blood cells. 3 mL of RPMI 1640 (10% FBS) medium was added to terminate lysis, centrifugation was performed at 400 g for 3 min, and the supernatant was discarded. Cells were cultured in a 10 mm culture dish using RPMI 1640 (10% FBS) medium with the addition of recombinant mouse GM-CSF (20 ng/mL) at 37° C. and 5% CO₂ for 7 days. On Day 3, the culture flask was gently shaken and the same volume of RPMI 1640 (10% FBS) medium containing GM-CSF (20 ng/mL) was replenished. On day 6, the medium was subjected to half-volume change treatment. On day 7, a small number of suspended and semi-adherent cells were collected. When the ratio of CD86⁺ CD80⁺ cells in CD11c⁺ cells was between 15-20% by flow cytometry, the induced cultured BMDC could be used for the next experiment.

### (4) Isolation and Expansion of Cancer Cell-Specific T Cells

Each C57BL/6 mouse was subcutaneously inoculated with 5×10⁵ B16F10 cells on the back on day 0, and the mice were subcutaneously injected with 100 μL of 1 mg PLGA nanoparticles on days 7, 14, 21, and 28, respectively. The mice were sacrificed on day 35, the tumor tissue of the mice was collected, the tumor tissue was cut into small pieces and digested with collagenase for 30 minutes, then a single-cell suspension was prepared through a cell strainer, and CD45⁺ CD3⁺ T cells were sorted from live cells in tumor tissue single-cell suspension (dead cells were labeled with live-dead cell dye to remove dead cells) using flow cytometry after centrifugation and washing. The BMDC (3 million cells) prepared in step (3) was co-incubated with nanoparticles (80 μg) or control nanoparticles (80 μg) loaded with the whole antigens of tumor tissue in 5 mL DMEM high glucose complete medium for 24 hours (37° C., 5% CO₂), then 50 thousand sorted T cells were added and co-incubated for additional 24 hours, and then the incubated CD3⁺ CD69⁺ T cells and CD3⁺ CD25⁺ T cells, i.e., cancer cell-specific T cells activated by whole-cell antigens of cancer cells, were sorted by flow cytometry. The cancer cell-specific T cells obtained by the above sorting were co-incubated with IL-2 (1000 U/mL), IL-7 (200 U/mL), IL-15 (200 U/mL), αCD-3 antibodies (10 ng/mL), and αCD-28 antibodies (10 ng/mL) in DMEM high glucose complete medium for 14 days (medium change every two days) to expand the isolated cancer cell-specific T cells.

### (5) Cancer Cell-Specific T Cells for Prevention of Cancer Metastasis

Female C57BL/6 aged 6-8 weeks were selected as model mice to prepare melanoma-bearing mice. One day before adoptive transfer of cells, the recipient mice were intraperitoneally injected with a 100 mg/kg dose of cyclophosphamide to eliminate immune cells in the recipient mice. The mice were injected intravenously with 100 μL of 4 million cancer cell-specific T cells on day 0. At the same time, each mouse was inoculated with 0.5×10⁵ B16F10 cells by intravenous injection on day 1, the mice were sacrificed on day 14, and the number of melanoma foci in the lungs of the mice was observed and recorded.

### (6) Experimental Results

As shown in FIG. 5, most of the melanoma foci of the mice grew up, while the T cell-pretreated mice had almost no cancer foci. Moreover, the effect of preventing melanoma lung metastasis of T cells isolated and expanded with assistance of nanoparticles loaded with whole-cell antigens of cancer cells was better than that of T cells isolated and expanded with assistance of nanoparticles loaded with four antigen peptides. This indicated that the nanoparticles loaded with whole-cell antigens of cancer cells can help isolate wider-spectrum and diverse cancer cell-specific T cells, so the number of T cell clones that can be obtained after expansion was wider-spectrum, and thus more cancer cells can be identified and killed, facilitating a better effect of preventing cancer metastasis.

### Example 5 Cancer Cell-Specific T Cells Isolated and Expanded with Assistance of Microparticles for Cancer Prevention

In this example, whole-cell antigens of cancer cells for B16F10 melanoma were first lysed using 6 M guanidine hydrochloride. Then, a microparticle system loaded with whole-cell antigens of cancer cells was prepared by using PLGA as a microparticle backbone material and CpG BW006 as an immune adjuvant. In this example, the method of silicification, and addition of cationic substances and anionic substances was employed to increase the antigen-loading capacity, and two rounds of silicification treatment were performed. After microparticles activated cancer cell-specific T cells, the activated cancer cell-specific T cells were isolated and expanded, and then injected into mice to prevent cancer.

### (1) Lysis of Cancer Cells

The cultured B16F10 melanoma cancer cell line was collected and centrifuged at 350 g for 5 minutes, then the supernatant was discarded and washed twice with PBS, and then the cancer cells were resuspended and lysed with 6 M guanidine hydrochloride. The whole-cell antigens of the cancer cells were lysed and solubilized in 6 M guanidine hydrochloride, which was the source of antigen raw material for preparing the microparticle system.

### (2) Preparation of a Microparticle System

In this example, the microparticles and the blank microparticles as controls were prepared by a double-emulsion method, and the double-emulsion method was properly modified and improved. In the preparation process of the microparticles, two modification methods, low-temperature silicification technology and addition of charged species, were adopted to increase the antigen-loading capacity. The molecular weight of the microparticle preparation material PLGA adopted was 38 KDa to 54 KDa, and the immune adjuvant adopted was CpG. The preparation method was as previously described. In the preparation process, the whole-cell antigens of cancer cells and adjuvant were first loaded inside the microparticles by the double-emulsion method, after the lysis components were loaded inside, 100 mg of the microparticles were centrifuged at 10000 g for 15 minutes, then the microparticles are resuspended using 7 mL of PBS and mixed with 3 mL of PBS solution containing a cell lysate (50 mg/mL), and then centrifuged at 10000 g for 20 minutes. The microparticles were then resuspended with 10 mL of a silicate solution (containing 120 mM NaCl, 100 mM tetramethyl orthosilicate and 1.0 mM HCl, pH 3.0) and fixed at room temperature for 12 h, centrifuged and washed with ultrapure water, then resuspended with 3 mL of PBS containing polyaspartic acid (10 mg/mL) for 10 min, then centrifuged at 10000 g for 15 min, resuspended with 10 mL of PBS containing the cell lysate (50 mg/mL) for reaction for 10 min, and then centrifuged at 10000 g for 20 min. Then 10 mL of a silicate solution (containing 150 mM NaCl, 80 mM tetramethyl orthosilicate and 1.0 mM HCl, pH 3.0) was used, and the microparticles were fixed at room temperature for 12 h, centrifuged and washed with ultrapure water and then resuspended with 3 mL of PBS containing histone (5 mg/mL) and polyarginine (10 mg/mL) for reaction for 10 min, then washed by centrifugation at 10000 g for 15 min, resuspended with 10 mL of PBS solution containing the cell lysate (50 mg/mL) for 10 min, then centrifuged at 10000 g for 15 min, and resuspended with 10 mL of ultrapure water containing 4% trehalose and then freeze-dried for 48 h; before use, the particles were resuspended in 7 mL of PBS and then added with 3 mL of adjuvant-containing cancer cell lysate components (protein concentration of 50 mg/mL) for reaction at room temperature for 10 min to obtain modified microparticles with the lysate both inside and outside undergoing two rounds of cryosilicification and addition of cationic and anionic species loaded. The average particle size of the microparticles was about 2.50 μm, and the surface potential of the microparticles was about −2 mV; and approximately 340 μg of protein or peptide components were loaded per 1 mg of PLGA microparticles, and a total of approximately 0.02 mg of the CpG immune adjuvant was used inside and outside per 1 mg of PLGA microparticles, with half inside and half outside.

The loaded whole-cell antigens of cancer cells were replaced with four melanoma antigen peptides of an equivalent amount of mass in the control microparticles, and the rest was the same as the microparticles loaded with the whole-cell antigens of cancer cells. The control microparticles used 0.02 mg of the adjuvant per 1 mg of PLGA microparticles, the particle size was about 2.50 μm, the surface potential of the microparticles was about −2 mV, and about 340 μg of the protein or peptide components were loaded per 1 mg of PLGA microparticles. The four loaded peptide neoantigens were B16-M20 (Tubb3, FRRKAFLHWYTGEAMDEMEFTEAESNM) (SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3), and TRP2:180-188 (SVYDFFVWL) (SEQ ID NO: 4).

The particle size of the blank microparticles was about 2.43 μm, and the surface potential was about −3 mV. When preparing the blank microparticles, 6 M guanidine hydrochloride containing an equivalent amount of CpG was adopted to replace the corresponding cell components.

### (3) Preparation of Dendritic Cells

This example illustrated how to prepare bone marrow-derived dendritic cells (BMDCs) by taking the preparation of dendritic cells from mouse bone marrow cells as an example. First, a 6-8-week-old C57 mouse was sacrificed by cervical dislocation. The tibia and femur of the hind leg were surgically removed and put into PBS, and the muscle tissue around the bone was removed cleanly with scissors and tweezers. Both ends of the bone were cut off with scissors, then a PBS solution was extracted with a syringe, needles were inserted from both ends of the bone into the bone marrow cavity, and the bone marrow was repeatedly rinsed into a culture dish. The bone marrow solution was collected, centrifuged at 400 g for 3 min, and then added with 1 mL of a red blood cell lysate to lyse red blood cells. 3 mL of RPMI 1640 (10% FBS) medium was added to terminate lysis, centrifugation was performed at 400 g for 3 min, and the supernatant was discarded. Cells were cultured in a 10 mm culture dish using RPMI 1640 (10% FBS) medium with the addition of recombinant mouse GM-CSF (20 ng/mL) at 37° C. and 5% CO₂ for 7 days. On Day 3, the culture flask was gently shaken and the same volume of RPMI 1640 (10% FBS) medium containing GM-CSF (20 ng/mL) was replenished. On day 6, the medium was subjected to a half-volume change treatment. On day 7, a small number of suspended and semi-adherent cells were collected. When the ratio of CD86⁺ CD80⁺ cells in CD11c⁺ cells was between 15-20% by flow cytometry, the induced cultured BMDC could be used for the next experiment.

### (4) Isolation and Expansion of Cancer Cell-Specific T Cells

On day 0, each C57BL/6 mouse was subcutaneously inoculated with 1.5×10⁵ B16F10 cells on the back. The mice were treated with radiation irradiation using radiation to irradiate the tumor site on day 10, day 15 and day 20. The mice were sacrificed on day 25, the tumor tissue of the mice in each group was collected, the tumor tissue of the mice was cut into small pieces and passed through a cell strainer to prepare a single-cell suspension, and then CD3⁺ T cells were sorted from live cells in the single-cell suspension of the tumor tissue using a magnetic bead sorting method (dead cells were labeled with live-dead cell dye to remove dead cells). The sorted T cells (0.5 million cells), the BMDC prepared in step (3) (5 million cells) and the microparticles (100 μg) were co-incubated in 2 mL of RPMI 1640 complete medium for 24 hours (37° C., 5% CO₂), and then CD69⁺ T cells in the T cells, i.e., cancer cell-specific T cells activated by whole-cell antigens of cancer cells, were sorted by the magnetic bead sorting method. The sorted cancer cell-specific T cells were co-incubated with IL-2 (2000 U/mL), αCD-3 antibodies (20 ng/mL) and αCD-28 antibodies (20 ng/mL) in RPMI 1640 complete medium for 7 days (medium change every two days) to expand the sorted cancer cell-specific T cells.

### (5) Expanded Cancer Cell-Specific T Cells for Cancer Prevention

Female C57BL/6 aged 6-8 weeks were selected as model mice to prepare melanoma-bearing mice. One day before adoptive transfer of cells, the recipient mice were intraperitoneally injected with a 100 mg/kg dose of cyclophosphamide to eliminate immune cells in the recipient mice. The mice were injected intravenously with 100 μL of 3 million cancer cell-specific T cells on day 0. At the same time, each mouse was inoculated with 1.5×10⁵ B16F10 cells subcutaneously on day 0, and the tumor volume size of the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (6) Experimental Results

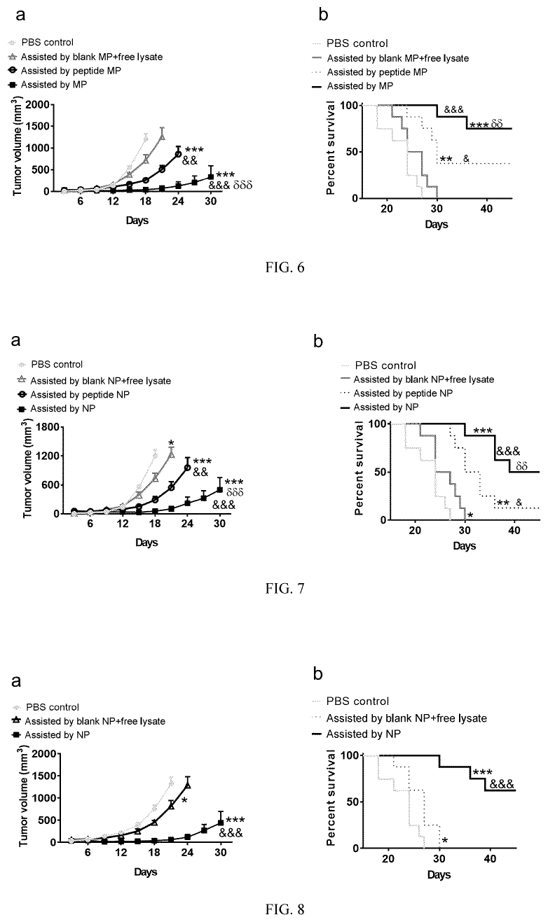

As shown in FIG. 6, the tumors of the mice in the control group all grew up, while the tumor growth rate of the mice treated with cancer cell-specific T cells significantly slowed down and the survival time was significantly prolonged. Moreover, the effect of preventing melanoma of T cells isolated and expanded with assistance of microparticles loaded with whole-cell antigens of cancer cells was better than that of T cells isolated and expanded with assistance of microparticles loaded with four antigen peptides. This indicated that the types of cancer cell-specific T cells that can be isolated with assistance of four neoantigen peptide microparticles were limited, so the number of T cell clones contained in the expanded T cell system was very small, and the cancer cells that can be recognized and killed were fewer. The microparticles loaded with the whole-cell antigens of cancer cells can help isolate more diverse cancer cell-specific T cells, so the number of T cell clones that can be obtained after expansion was wider-spectrum, and the more cancer cells that can be identified and killed, the better the effect of treating or preventing cancer.

### Example 6 Cancer Cell-Specific T Cells for Cancer Prevention

In this example, B16F10 melanoma tumor tissue was first lysed using 8 M urea, and the lysate components of the tumor tissue were solubilized. Then, a nanoparticle system loaded with whole-cell antigens of cancer cells was prepared with PLGA as a nanoparticle backbone material, and Poly(I:C) and CpG2006 as immune adjuvants, cancer cell-specific T cells in tumor-infiltrating lymphocytes were activated and isolated in vitro by using nanoparticles and antigen-presenting cells, and the above cells were expanded for cancer prevention.

### (1) Collection and Lysis of Tumor Tissue

Each C57BL/6 mouse was subcutaneously inoculated with 1.5×10⁵ B16F10 cells on the back, and the mice were sacrificed and the tumor tissue removed when the tumors each grew to a volume of approximately 1000 mm³. The tumor tissue was cut into pieces and ground, the cells were lysed by adding an appropriate amount of 8 M urea through a cell strainer, and the cell lysate was solubilized. The above was the source of antigen raw materials for preparing the nanoparticle system.

### (2) Preparation of a Nanoparticle System

In this example, the nanoparticles and the blank nanoparticles as controls were prepared by the solvent evaporation method. The molecular weight of the nanoparticle preparation material PLGA adopted was 7 KDa to 17 KDa, the immune adjuvants adopted were poly(I:C) and CpG2006, and the lysate components and adjuvants were encapsulated inside the nanoparticles. The preparation method was as previously described. In the preparation process, the lysate components and adjuvants were first loaded inside the nanoparticles by a double-emulsion method. After the antigen lysate components and adjuvants were loaded inside, 100 mg nanoparticles were centrifuged at 12000 g for 20 minutes and freeze-dried for 48 h after resuspension using 10 mL of ultrapure water containing 4% trehalose to obtain a freeze-dried powder for later use. The average particle size of the nanoparticles was about 270 nm, and the surface potential of the nanoparticles was about −3 mV; and approximately 110 μg of the protein or peptide components were loaded per 1 mg of PLGA nanoparticles, and 0.02 mg each of poly(I:C) and CpG2006 immune adjuvants were used per 1 mg of PLGA nanoparticles. The particle size of the blank nanoparticles was about 250 nm, and 8 M urea containing an equivalent amount of poly(I:C) and CpG2006 was used instead of the lysate components in the preparation of the blank nanoparticles. The lysate components were replaced with four melanoma antigen peptides of an equivalent amount of mass in the control nanoparticles, and the rest was the same as the nanoparticles loaded with the whole-cell antigens of cancer cells. The control nanoparticles used 0.02 mg each of poly(I:C) and CpG2006 immune adjuvants per 1 mg of PLGA nanoparticles, the particle size was about 270 nm, the nanoparticle surface potential was about −3 mV, and about 110 μg of peptide component was loaded per 1 mg of PLGA nanoparticles. The four loaded peptide neoantigens were B16-M20 (Tubb3, FRRKAFLHWYTGEAMDEMEFTEAESNM) (SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3), and TRP2:180-188 (SVYDFFVWL) (SEQ ID NO: 4).

### (3) Isolation and Expansion of Cancer Cell-Specific T Cells

On day 0, each C57BL/6 mouse was subcutaneously inoculated with 1.5×10⁵ B16F10 cells on the back, and 100 μL of αPD-1 antibodies (10 mg/kg) were subcutaneously injected into the mice on days 8, 10, 12, 14, 16, 18, and 20, respectively. The mice were sacrificed on day 24, and the tumor tissue of the mice in each group was collected respectively to prepare a single-cell suspension of the tumor tissue, and then CD3⁺ T cells were sorted from live cells in the single-cell suspension of the tumor tissue using magnetic bead sorting method (dead cells were labeled with a live-dead cell dye to remove dead cells). Then, the sorted T cells (0.5 million cells) were co-incubated with allogeneic B cells (2.5 million cells), and nanoparticles loaded with whole-cell antigens of tumor tissue (100 μg) or control nanoparticles (100 μg) in 10 mL RPMI 1640 complete medium for 48 hours (37° C., 5% CO₂), and then the incubated CD3⁺ CD8⁺ CD69⁺ T cells and CD3⁺ CD4⁺ CD69⁺ T cells, i.e., cancer cell-specific T cells activated by whole-cell antigens of cancer cells, were sorted by flow cytometry. The sorted cancer cell-specific T cells were co-incubated with IL-2 (2000 U/mL), αCD-3 antibodies (20 ng/mL), and αCD-28 antibodies (20 ng/mL) in RPMI 1640 complete medium for 11 days (medium change every two days) to expand the sorted cancer cell-specific T cells.

### (4) Cancer Cell-Specific T Cells for Cancer Prevention

Female C57BL/6 aged 6-8 weeks were selected as model mice to prepare melanoma-bearing mice. One day before the transplantation of mouse cancer cell-specific T cells, the recipient mice were intraperitoneally injected with a 100 mg/kg dose of cyclophosphamide to eliminate immune cells in the recipient mice. The mice were subcutaneously injected with 100 μL of 0.8 million expanded cancer cell-specific CD8⁺ T cells and 0.2 million expanded cancer cell-specific CD4⁺ T cells on day 0. At the same time, each mouse was inoculated with 1.5×10⁵ B16F10 cells subcutaneously on day 0, and the tumor volume size of the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (5) Experimental Results

As shown in FIG. 7, the tumors of the control mice all grew up, while the tumor growth rate of the mice transplanted with cancer cell-specific T cells obtained by nanoparticles loaded with whole-cell antigens of cancer cells was significantly slower, and the tumors of most mice disappeared after cancer cell inoculation. The effect of preventing melanoma of T cells isolated and expanded with assistance of nanoparticles loaded with whole-cell antigens of cancer cells was better than that of T cells isolated and expanded with assistance of nanoparticles loaded with four antigen peptides, which indicated that the types of cancer cell-specific T cells that can be isolated with assistance of four neoantigen peptide nanoparticles were limited, so the number of T cell clones contained in the expanded T cell system was very small, and the cancer cells that can be recognized and killed were fewer. The nanoparticles loaded with the whole-cell antigens of cancer cells can help isolate more diverse cancer cell-specific T cells, so the number of T cell clones that can be obtained after expansion was wider-spectrum, and the more cancer cells that can be identified and killed, the better the effect of treating or preventing cancer.

### Example 7 Cancer Cell-Specific T Cells for Colon Cancer Treatment

This example used MC38 mouse colon cancer as a cancer model to illustrate how nanoparticles were used to assist in isolating broad-spectrum cancer cell-specific T cells for the treatment of colon cancer. Colon cancer tumor tissue and lung cancer cells were first lysed to prepare a water-soluble antigen mixture (mass ratio 1:1) and a water-insoluble antigen mixture (mass ratio 1:1) mixture, and the water-soluble antigen mixture and the water-insoluble antigen mixture were mixed at a mass ratio of 1:1. Then, PLA was used as a nanoparticle backbone material, CpGM362 and Bacille Calmette-Guerin (BCG) were used as immune adjuvants to prepare nanoparticles, and the nanoparticles were used to activate cancer cell-specific T cells in vitro, and then the cancer cell-specific T cells were isolated, extracted and expanded for the treatment of colon cancer.

### (1) Lysis and Component Collection of Tumor Tissue and Cancer Cells

Each C57BL/6 mouse was subcutaneously inoculated with 2×10⁶ MC38 cells on the back, and the mice were sacrificed and the tumor tissue removed when the tumors each grew to a volume of approximately 1000 mm³. The tumor tissue was cut into pieces and ground, the sample was filtered through a cell filter strainer and an appropriate amount of pure water was added, and freezing-thawing cycle was repeated 5 times, accompanied by ultrasound to destroy and lyse the cells. After the cells were lysed, the lysate was centrifuged at a rotational speed greater than 5000 g for 5 minutes, and the supernatant was taken to obtain water-soluble antigens that were soluble in pure water; and adding 8 M urea to the obtained precipitated part to solubilize the precipitated part can convert the water-insoluble antigens that were insoluble in pure water into soluble ones in an 8 M aqueous urea solution.

The cultured LLC lung cancer cell lines were harvested and centrifuged at 350 g for 5 minutes, then the supernatant was discarded and washed twice with PBS, and then the cells were resuspended with ultrapure water and frozen and thawed repeatedly 5 times, which can be accompanied by ultrasound to destroy and lyse the cells. After the cells were lysed, the lysate was centrifuged at a rotational speed of 3000 g for 6 minutes, and the supernatant was taken to obtain water-soluble antigens that were soluble in pure water; and adding 8 M urea to the obtained precipitated part to solubilize the precipitated part can convert the water-insoluble antigens that were insoluble in pure water into soluble ones in an 8 M aqueous urea solution.

Water-soluble antigens from colon cancer tumor tissue and lung cancer cells were mixed at a mass ratio of 1:1; and the water-insoluble antigens solubilized in 8 M urea were also mixed at a mass ratio of 1:1. Then, the water-soluble antigen mixture and the water-insoluble antigen mixture were mixed at a mass ratio of 1:1, and the mixture was the source of raw materials for preparing the nanoparticles.

### (2) Lysis and Component Collection of BCG

The lysis method and component collection method of BCG were the same as the lysis method and component collection method of cancer cells, the water-soluble antigens and the solubilized water-insoluble antigens were mixed in a mass ratio of 1:1.

### (3) Preparation of Nanoparticles

In this example, the nanoparticles were prepared by a solvent evaporation method. The molecular weight of the nanoparticle preparation material PLA adopted was 20 KDa, the immune adjuvants adopted were CpGM362 and BCG, and the adjuvants were distributed inside and on the surface of the nanoparticles at the same time. The preparation method was as previously described. In the preparation process, the lysate mixture and adjuvants were first loaded inside the nanoparticles by a double-emulsion method. After the lysate and adjuvants were loaded inside, 100 mg nanoparticles were centrifuged at 10000 g for 20 minutes and freeze-dried for 48 h after resuspension using 10 mL of ultrapure water containing 4% trehalose. 20 mg of nanoparticles were resuspended in 0.9 mL of PBS before use and incubated in 0.1 mL of a sample containing the lysate mixture (80 mg/mL) and adjuvants at room temperature for 5 minutes before use. The average particle size of the nanoparticles was about 280 nm, and the surface potential of the nanoparticles was about −3 mV; and approximately 140 μg of the protein or peptide components were loaded per 1 mg of PLGA nanoparticles, and each 1 mg of PLGA nanoparticles contained 0.04 mg of CpGM362 and BCG immune adjuvants. The particle size of the blank nanoparticles was about 260 nm, and a solution containing an equivalent quantity of adjuvants was adopted to replace the corresponding lysate components when preparing the blank nanoparticles.

### (4) Isolation and Expansion of Cancer Cell-Specific T Cells

Each C57BL/6 mouse was subcutaneously inoculated with 1.5×10⁵ MC38 cells on the back on day 0, and the mice were subcutaneously injected with 100 μL of 1 mg PLGA nanoparticles on days 10, 15 and 21, respectively. The mice were sacrificed on day 24, tumor tissue of the mice was collected, tumor tissue single-cell suspensions were prepared and T cells were sorted from the live cells (dead cells were labeled with a viable dead cell dye to remove dead cells) using a magnetic bead method. T cells (0.4 million cells), B cells (0.4 million cells), macrophages (0.4 million cells), and nanoparticles (40 μg) loaded with all components of the tumor tissue were incubated in DMEM complete medium for 96 hours, and then the incubated CD3⁺ CD8⁺ CD69⁺ T cells, that is, cancer cell-specific T cells activated by whole-cell antigens of cancer cells, were sorted by flow cytometry. The sorted cancer cell-specific T cells were co-incubated with IL-2 (1000 U/mL), IL-7 (200 U/mL), IL-15 (200 U/mL), and αCD-3 antibodies (10 ng/mL) in DMEM complete medium for 8 days (medium change every two days) to expand the sorted cancer cell-specific CD8⁺ T cells.

### (5) Cancer Cell-Specific CD8⁺ T Cells for Cancer Treatment

Female C57BL/6 aged 6-8 weeks were selected as model mice to prepare colon cancer tumor-bearing mice. Each mouse was subcutaneously inoculated with 2×10⁶ MC38 cells on day 0 and injected with 100 μL containing 2 million cancer cell-specific CD8⁺ T cells on days 4, 7, 10, 15, and 20, respectively. The size of tumor volume in the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (6) Experimental Results

As shown in FIG. 8, the tumors of the mice in the PBS control group and the blank nanoparticle control group grew rapidly and the survival time of the mice was short. Compared with the control group, the tumor growth rate of the mice in the cancer cell-specific T cell transplantation group isolated and expanded with assistance of nanoparticles was significantly slower, and the tumors of some mice disappeared and recovered. In summary, the immune cell therapy regimen of the present disclosure had a good therapeutic effect on colon cancer.

### Example 8 Tumor-Infiltrating T Cells Isolated with Assistance of Nanoparticles for Melanoma Treatment

This example used melanoma as a cancer model to illustrate how nanoparticles loaded with whole-cell antigens of cancer cells derived from melanoma and lung cancer tumor tissue can be used to assist in isolating cancer cell-specific T cells in tumor-infiltrating lymphocytes and treat melanoma with the cells. In this example, B16F10 melanoma tumor tissue and LLC lung cancer tumor tissue were first lysed to prepare a water-soluble antigen mixture (mass ratio 3:1) and a water-insoluble antigen mixture (3:1) of the tumor tissue. PLGA was used as a nanoparticle skeleton material, and manganese particles and CpG2395 were used as immune adjuvants to prepare nanoparticles loaded with the above mixture, and then the nanoparticles were used as to activate cancer cell-specific T cells in tumor-infiltrating lymphocytes, and the above cells were isolated and expanded for cancer treatment.

### (1) Lysis and Component Collection of Tumor Tissue

Each C57BL/6 mouse was subcutaneously inoculated with 1.5×10⁵ B16F10 cells or 2×10⁶ LLC lung cancer cells on the back, and the mice were sacrificed and the tumor tissue removed when the tumors each grew to a volume of approximately 1000 mm³. The lysis and component collection methods of the tumor were the same as in Example 1. The water-soluble antigens from melanoma tumor tissue and lung cancer tumor tissue and the original water-insoluble antigens solubilized in 8 M urea were mixed in a ratio of 3:1, respectively, which was the source of antigens for preparing the nanoparticles.

### (2) Preparation of Nanoparticles

The nanoparticles in this example were prepared by a double-emulsion method. At the time of preparation, the nanoparticles loaded with the water-soluble antigens in the whole-cell antigens of cancer cells and the nanoparticles loaded with the water-insoluble antigens in the whole-cell antigens of cancer cells were respectively prepared and then used together when used. The molecular weight of nanoparticle preparation material PLGA adopted was 24 KDa to 38 KDa, and the immune adjuvants adopted were manganese colloidal particles and CpG2395. Firstly, the manganese adjuvant was prepared, and then the manganese adjuvant was mixed with the water-soluble antigens or the water-insoluble antigens in the whole-cell antigens of cancer cells as the first aqueous phase to prepare the nanoparticles loaded with the antigen and the adjuvant internally by a double-emulsion method. When preparing the manganese adjuvant, 1 mL of a 0.3 M Na₃PO₄ solution was first added to 9 mL of normal saline, then added with 2 mL of a 0.3 M MnCl₂ solution, and allowed to stand overnight to obtain a Mn₂OHPO₄ colloidal manganese adjuvant, with the particle size of the manganese adjuvant of about 13 nm. Then, the manganese adjuvant was mixed with the water-soluble antigens (60 mg/mL) or the water-insoluble antigens (60 mg/mL) in the whole-cell antigens of cancer cells at a volume ratio of 1:3, and the antigens and manganese adjuvant were loaded into the nanoparticles by the double-emulsion method. After internal loading of the antigens (lysis components) and adjuvant, 100 mg of nanoparticles were centrifuged at 10000 g for 20 min, resuspended with 10 mL of ultrapure water containing 4% trehalose and freeze-dried for 48 h for later use. The average particle size of the nanoparticles was about 370 n, and the surface potential of the nanoparticles was about −5 mV; and approximately 120 μg of the protein or peptide components were loaded per 1 mg of PLGA nanoparticles, and 0.04 mg of CpG2395 adjuvant was used per 1 mg of PLGA nanoparticles.

The particle size of the blank nanoparticles was about 310 n. When preparing the blank nanoparticles, pure water or 8 M urea containing an equivalent quantity of the manganese adjuvant and CpG2395 adjuvant was used to replace the corresponding water-soluble antigens and water-insoluble antigens.

### (3) Preparation of Dendritic Cells

Same as Example 4.

### (4) Isolation and Expansion of Cancer Cell-Specific T Cells

1.5×10⁵ B16F10 cells were subcutaneously inoculated on the back of each C57BL/6 mouse on day 0, and 100 μL of 1 mg PLGA nanoparticles loaded with water-soluble antigens and 100 μL of 1 mg PLGA nanoparticles loaded with water-insoluble antigens were subcutaneously injected in the mice on days 10, 15 and 20, respectively. The mice were sacrificed on day 24, tumor tissue of the mice was collected, tumor tissue single-cell suspensions were prepared and CD3⁺ T cells were sorted from the live cells (dead cells were labeled with a viable dead cell dye to remove dead cells) using a magnetic bead method. T cells (0.3 million cells), BMDC (3 million cells), and nanoparticles loaded with tumor tissue water-soluble antigens (60 μg) and nanoparticles loaded with insoluble antigens (60 μg) were co-incubated in 3 mL RPMI 1640 complete medium for 96 hours (37° C., 5% CO₂), and then the incubated CD3⁺ CD69⁺ T cells, i.e., cancer cell-specific T cells activated by whole-cell antigens of cancer cells, were sorted by flow cytometry. The sorted cancer cell-specific T cells were co-incubated with IL-2 (4000 U/mL) and αCD-3 antibodies (20 μg) in RPMI 1640 complete medium for 12 days (medium change every two days) to expand the sorted cancer cell-specific T cells.

### (5) Cancer Cell-Specific T Cells for Cancer Treatment

Female C57BL/6 aged 6-8 weeks were selected as model mice to prepare melanoma-bearing mice. Each mouse was inoculated subcutaneously with 1.5×10⁵ B16F10 cells on day 0, and the mice were injected with 100 μL containing 2 million cancer cell-specific T cells on days 4, 7, 10, 15, and 20, respectively. The size of tumor volume in the mice was recorded every 3 days from day 3. The tumor volume was calculated using the formula v=0.52×a×b², where v was the tumor volume, a was the tumor length, and b was the tumor width. When the tumor volume of the mouse exceeded 2000 mm³ in the mouse survival test, the mouse was regarded as dead and the mouse was euthanized.

### (6) Experimental Results

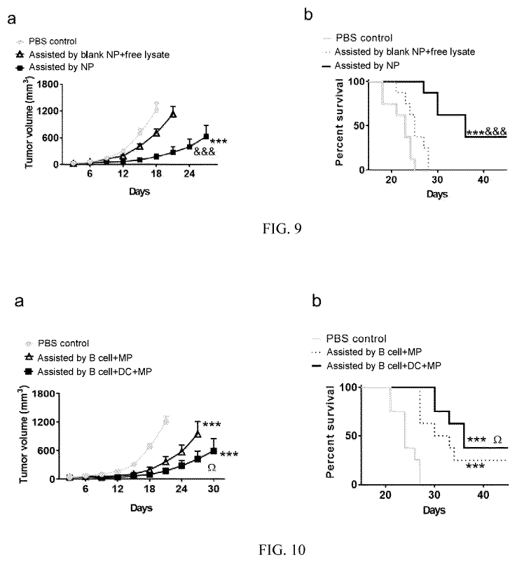

As shown in FIG. 9, the tumors of the mice in the PBS control group and the blank nanoparticle control group grew rapidly. Compared with the control group, the tumor growth rate of the mice in the tumor-infiltrating T cell transplantation group isolated with assistance of nanoparticles was significantly slower, and the tumors of some mice disappeared and recovered. In summary, the cell therapy regimen of the present disclosure had a therapeutic effect on melanoma.

### Example 9 Cancer Cell-Specific T Cells for Breast Cancer Prevention

This example used 4T1 mouse triple-negative breast cancer as a cancer model to illustrate how to use 8 M urea to lyse whole-cell antigens of cancer cells and prepare a microparticle system loaded with whole-cell antigens of cancer cells, and used the microparticle to assist in isolating cancer cell-specific T cells from tumor tissue infiltration and use for prevention of breast cancer.

### (1) Lysis of Cancer Cells

The cultured 4T1 cells were centrifuged at 400 g for 5 minutes, then washed twice with PBS and resuspended in ultrapure water. The obtained cancer cells were inactivated and denatured by ultraviolet rays and high-temperature heating, respectively, and then the breast cancer cells were lysed with an appropriate amount of 8 M urea and the lysate was solubilized, which was the source of raw materials for preparing the particle system.

### (2) Preparation of a Microparticle System

In this example, the microparticles were prepared by a double-emulsion method, the molecular weight of the microparticle skeleton material PLGA was 38 KDa to 54 KDa, and the immune adjuvants adopted were CpG2395 and Poly(I:C). During preparation, the microparticles internally loaded with the lysate components and adjuvants were prepared by the double-emulsion method. After the internal loading of the lysate and adjuvants, 100 mg of the microparticles were centrifuged at 9000 g for 20 minutes, and resuspended using 10 mL of ultrapure water containing 4% trehalose and then dried for 48 h for later use. The average particle size of the microparticle system was about 2.1 μm, and the surface potential was about −6 mV; and each 1 mg of PLGA microparticles was loaded with approximately 110 μg of the protein or peptide components, containing 0.03 mg each of CpG2395 and Poly(I:C).

### (3) Preparation of Dendritic Cells

Same as Example 4.

### (4) Isolation and Expansion of Cancer Cell-Specific T Cells

Each BALB/c mouse was subcutaneously inoculated with 1×10⁶ 4T1 cells on the back on day 0, and the mice were subcutaneously injected with 100 L of 1 mg PLGA microparticles on days 10, 17, and 24, respectively. The mice were sacrificed on day 30, and the tumor tissue and spleen of the mice were collected to prepare a tumor tissue single-cell suspension and a splenocyte single-cell suspension. CD3⁺ T cells were isolated from live cells (dead cells were labeled with live-dead cell dye to remove dead cells) in the tumor tissue single-cell suspension using flow cytometry; and CD19⁺ B cells were isolated from live cells (dead cells were labeled with live-dead cell dye to remove dead cells) in splenocytes. T cells (0.1 million cells), B cells (3 million cells), BMDC (2 million cells), and microparticles (20 μg) were incubated in 2 mL DMEM complete medium for 72 hours (37° C., 5% CO₂), and then CD3⁺ CD69⁺ T cells, i.e., cancer cell-specific T cells activated by whole-cell antigens of cancer cells, were sorted therefrom by flow cytometry; or T cells (0.1 million cells), B cells (5 million) and microparticles (20 μg) are incubated in 2 mL DMEM complete medium for 72 hours (37° C., 5% CO₂), and then CD3⁺ CD69⁺ T cells, i.e. cancer cell-specific T cells activated by whole-cell antigens of cancer cells, were sorted therefrom by flow cytometry. The cancer cell-specific T cells sorted by the above two solutions were incubated with IL-2 (4000 U/mL) and αCD-3 antibodies (20 ng/mL) in DMEM complete medium for 12 days (medium change every two days) to expand the sorted cancer cell-specific T cells.

### (5) Cancer Cell-Specific T Cells for Cancer Prevention

Female BALB/c at 6-8 weeks were selected as model mice to prepare breast cancer tumor-bearing mice. One day before the adoptive transfer of cells, the recipient mice were intraperitoneally injected with a 100 mg/kg dose of cyclophosphamide to eliminate immune cells in the recipient mice. The mice were injected subcutaneously with 100 μL of 1.5 million expanded cancer cell-specific T cells on day 0. At the same time, each mouse was inoculated with 1×10⁶ 4T1 cells subcutaneously on day 0, and the tumor volume size of the mice was recorded every 3 days from day 3. The method of monitoring mouse tumors is the same as above.

### (6) Experimental Results

As shown in FIG. 10, compared with the control group, the tumor growth rate of the mice treated with cancer cell-specific T cells without microparticle-assisted isolation was significantly slower and the survival time was significantly prolonged. Moreover, using two kinds of antigen-presenting cells simultaneously in the process of isolating cancer cell-specific T cells was better than using only one kind of antigen-presenting cells. It can be seen that the T cells of the present disclosure had a preventive effect on breast cancer.

### Example 10 Cancer Cell-Specific T Cells for Cancer Metastasis Prevention

This example illustrated the use of nanoparticle-assisted isolation of cancer cell-specific T cells from tumor tissue-infiltrating lymphocytes for prevention of cancer metastasis using a mouse melanoma and mouse lung metastasis cancer model. In actual application, the specific dosage form, adjuvant, dosing time, dosing frequency, and dosing regimen can be adjusted according to situations. In this example, mouse melanoma tumor tissue and cancer cells were lysed with 8 M urea and then lysed, and then the tumor tissue lysis components and the cancer cell lysis components were loaded on a nanoparticle system at a mass ratio of 1:2, and the particles were used to assist in isolating cancer cell-specific T cells in tumor tissue-infiltrating lymphocytes to prevent cancer metastasis in mice. In this example, nanoparticles loaded with four peptide neoantigens B16-M20 (Tubb3, FRRKAFLHWYTGEAMDEMEFTEAESNM) (SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3) and TRP2:180-188(SVYDFFVWL) (SEQ ID NO: 4) were used as control nanoparticles.

### (1) Lysis of Tumor Tissue and Cancer Cells

After collecting mouse B16F10 melanoma tumor tissue and cultured cancer cells, whole-cell antigens of cancer cells from the tumor tissue and cancer cells were lysed and solubilized with 8 M urea, and then the tumor tissue components and the cancer cell components were miscible at a mass ratio of 1:2.

### (2) Preparation of Nanoparticles

In this example, the nanoparticles were prepared by a solvent evaporation method, the molecular weight of the nanoparticle preparation material PLGA adopted was 24 KDa to 38 KDa, and no immune adjuvant was used. The preparation method was as previously described. In the preparation process, the cell components were first loaded inside the nanoparticles by the double-emulsion method, and after the lysis components were loaded inside, 100 mg nanoparticles were centrifuged at 10000 g for 20 minutes and freeze-dried for 48 h after resuspension using 10 mL of ultrapure water containing 4% trehalose for later use. The average particle size of the nanoparticles was about 270 nm; and approximately 90 μg of the protein and peptide components were loaded per 1 mg of PLGA nanoparticles. The preparation method of control nanoparticles loaded with four antigen peptides was the same as above. The particle size of the control nanoparticles was about 260 nm, and about 90 μg of antigen peptides were loaded per 1 mg of PLGA nanoparticles. The blank control nanoparticles were not loaded with any cell components.

### (3) Isolation and Expansion of Cancer Cell-Specific T Cells

Each C57BL/6 mouse was subcutaneously inoculated with 1.5×10⁵ B16F10 cells on the back on day 0, and the mice were subcutaneously injected with 100 μL of 1 mg PLGA nanoparticles on days 14 and 24, respectively. The mice were sacrificed on day 26 and the tumor tissue of the mice was harvested. A single-cell suspension of the tumor tissue and a single-cell suspension of the splenocytes were prepared respectively. CD45⁺ lymphocytes were isolated from tumor-infiltrating live cells (dead cells were labeled with live-dead cell dye to remove dead cells) in the tumor tissue single-cell suspension using flow cytometry; and CD19⁺ B cells and CD11c⁺ DC cells were isolated from live cells (dead cells were labeled with live-dead cell dye to remove dead cells) in splenocytes using flow cytometry. CD45+ cells (1 million cells), B cells (2 million cells) and DC cells (0.4 million cells) were co-incubated with nanoparticles loaded with whole-cell antigens of cancer cells (80 μg) or 80 μg control nanoparticles (or 80 μg blank nanoparticles+free lysate) in high-glucose DMEM complete medium for 72 h (37° C., 5% CO₂). CD3⁺ CD137⁺ T cells, i.e. cancer cell-specific T cells activated by whole-cell antigens of cancer cells, were then isolated from the incubated cells by flow cytometry. The isolated cancer cell-specific T cells described above were co-incubated with IL-2 (2000 U/mL) and αCD-3 antibodies (20 ng/mL) in high glucose DMEM complete medium for 18 days (medium change every 2 days) to expand cancer cell-specific T cells.

### (4) Cancer Cell-Specific T Cells for Prevention of Cancer Metastasis

Female C57BL/6 aged 6-8 weeks were selected as model mice to prepare melanoma-bearing mice. One day before the adoptive transfer of cells, the recipient mice were intraperitoneally injected with a 100 mg/kg dose of cyclophosphamide to eliminate immune cells in the recipient mice. The mice were injected intravenously with 100 μL containing 2 million isolated and expanded cancer cell-specific T cells on day 0. At the same time, each mouse was inoculated with 0.5×10⁵ B16F10 cells by intravenous injection on day 1, the mice were sacrificed on day 14, and the number of melanoma foci in the lungs of the mice was observed and recorded.

### (5) Experimental Results

As shown in FIG. 11, the cancer cell-specific T cells isolated and expanded with assistance of nanoparticles can effectively prevent cancer metastasis. Moreover, the cancer cell-specific T cells isolated with assistance of nanoparticles loaded with whole-cell antigens of cancer cells had a better effect of preventing cancer metastasis than the cancer cell-specific T cells isolated with assistance of loaded with peptide antigens alone.

### Example 11 Tumor Tissue-Infiltrating Cancer Cell-Specific T Cells for Pancreatic Cancer Treatment

In this example, mouse Pan02 pancreatic cancer tumor tissue and MC38 colon cancer tumor tissue lysis components were loaded onto nanoparticles at a ratio of 3:1, and cancer cell-specific T cells in tumor-infiltrating lymphocytes were isolated after activation using the nanoparticles, and then expanded to treat pancreatic cancer. In the experiment, the tumor tissue of mice with pancreatic cancer and colon cancer was first obtained and lysed to prepare water-soluble antigens and original water-insoluble antigens solubilized in 6 M guanidine hydrochloride. In preparing the particles, PLGA was used as a nanoparticle backbone material and BCG was used as an adjuvant to prepare the nanoparticles, and then the nanoparticles were used to assist in isolating cancer cell-specific T cells from tumor-infiltrating lymphocytes.

### (1) Lysis and Component Collection of Tumor Tissue

Each C57BL/6 mouse was subcutaneously inoculated with 2×10⁶ MC38 colon cancer cells or 1×10⁶ Pan02 pancreatic cancer cells under the axillary area, and the mice were sacrificed and the tumor tissue removed when the inoculated tumors in each mouse grew to a volume of about 1000 mm³. The lysis method and the collection method of each component were the same as in Example 1, except that 6 M guanidine hydrochloride was used instead of 8M urea to solubilize the water-insoluble antigens. The water-soluble antigens were a 3:1 mixture of water-soluble antigens of the pancreatic cancer tumor tissue and water-soluble antigens of the colon cancer tumor tissue; and the water-insoluble antigens were a 3:1 mixture of water-insoluble antigens of the pancreatic cancer tumor tissue and water-insoluble antigens of the colon cancer tumor tissue. The water-soluble antigen mixture and the water-insoluble antigen mixture were mixed at a mass ratio of 1:1. The lysis and dissolution method of BCG was the same as the lysis method of the tumor tissue. The water-soluble antigens and the water-insoluble antigens were mixed at a mass ratio of 1:1.

### (2) Preparation of Nanoparticles

The nanoparticles in this example were prepared by a double-emulsion method. The molecular weight of the nanoparticle preparation material PLGA adopted was 7 KDa to 17 KDa, the immune adjuvant adopted was BCG, and the BCG was encapsulated inside the nanoparticles. The preparation method was as previously described. In the preparation process, the lysate components and adjuvants were first loaded inside the nanoparticles by a double-emulsion method. After the antigen lysate components and adjuvants were loaded inside, 100 mg nanoparticles were centrifuged at 12000 g for 20 minutes and freeze-dried for 48 h after resuspension using 10 mL of ultrapure water containing 4% trehalose to obtain a freeze-dried powder for later use. 20 mg of nanoparticles were solubilized in 0.9 mL of PBS before nanoparticle injection, mixed with 0.1 mL of a sample containing the lysate (80 mg/mL) and used after 10 min at room temperature. The average particle size of the nanoparticles was about 260 nm, and the surface potential of the nanoparticles was about −4 mV; and approximately 130 g of the protein and peptide components were loaded per 1 mg of PLGA nanoparticles, and 0.08 mg of BCG immune adjuvant was used per 1 mg of PLGA nanoparticles. The particle size of the blank nanoparticles was about 250 nm and contained an equivalent quantity of the adjuvant.

### (3) Isolation and Expansion of Cancer Cell-Specific T Cells

Each C57BL/6 mouse was subcutaneously inoculated with 2×10⁶ Pan02 cells on the back on day 0, and the mice were subcutaneously injected with 100 μL of 2 mg PLGA nanoparticles on days 12, 15, 20, and 27, respectively. The mice were sacrificed on day 30, and the tumor tissue and spleen of the mice were harvested to prepare a single-cell suspension of the tumor tissue and splenocytes. The isolation of CD45⁺ CD3⁺ T cells in tumor-infiltrating lymphocytes and the isolation of B cells in splenocytes were the same as in Example 3. B cells (2 million cells), DC2.4 cells (1 million cells), bone marrow-derived macrophages (BMDM, 1 million cells), and T cells (0.5 million cells) were co-incubated with nanoparticles loaded with the whole antigens of tumor tissue (100 μg) or blank nanoparticles (100 μg)+free lysate in DMEM high glucose medium for 48 h (37° C., 5% CO₂). CD3⁺ CD69⁺ T cells, i.e. cancer cell-specific T cells activated by whole-cell antigens of cancer cells, were then sorted from the incubated cells by flow cytometry. The cancer cell-specific T cells obtained by the above sorting were co-incubated with IL-2 (2000 U/mL) and αCD-3 antibodies (30 ng/mL) in high-glucose DMEM medium for 15 days (medium exchange every two days) to expand cancer cell-specific T cells.

### (4) Cancer Cell-Specific T Cells for Cancer Treatment

Female C57BL/6 aged 6-8 weeks were selected as model mice to prepare pancreatic cancer tumor-bearing mice. Each mouse was inoculated subcutaneously with 1×10⁶ Pan02 cells on day 0, and the mice were injected with 100 μL containing 2 million cancer cell-specific T cells on days 4, 7, 10, 15, 20, and 25, respectively. The size of tumor volume in the mice was recorded every 3 days from day 3. The tumor volume calculation method and the mouse survival monitoring method were the same as above.

### (5) Experimental Results

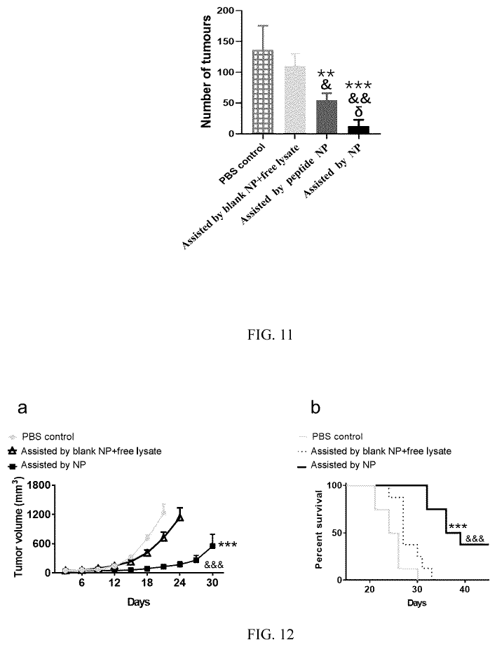

As shown in FIG. 12, the cancer cell-specific T cells isolated from tumor-infiltrating lymphocytes with nanoparticle assistance can be effective in treating pancreatic cancer.

### Example 12 Cancer Cell-Specific T Cells for Cancer Prevention

This example used mannose as the target to illustrate how actively targeting nanoparticles were used to assist in isolating cancer cell-specific T cells in tumor tissue-infiltrating lymphocytes and for cancer prevention. In actual application, the specific dosage form, adjuvant, dosing time, dosing frequency, and dosing regimen can be adjusted according to situations. The nanoparticle system can be absorbed into dendritic cells through mannose receptors on the surface of dendritic cells, and then activate cancer cell-specific T cells. The isolated T cells can be used for cancer prevention after expansion.

### (1) Lysis of Cancer Cells

The cultured B16F10 cancer cells were collected, and then 8 M urea was adopted to lyse and solubilize whole-cell antigens of cancer cells derived from cancer cells.

### (2) Preparation of a Nanoparticle System

The nanoparticle system in this example was prepared using a double-emulsion method. The nanoparticle preparation materials adopted were PLGA and mannose-modified PLGA, both of which had molecular weights of 7 KDa to 17 KDa. When the nanoparticles with target heads were prepared, the mass ratio of the two when used together was 4:1. The immune adjuvants used were Poly(I:C) and CpG SL03. The preparation method was as described previously. The lysate components and adjuvants were co-loaded inside the nanoparticles by the double-emulsion method, and then 100 mg of the nanoparticles were centrifuged at 10000 g for 20 minutes, resuspended with 10 mL of ultrapure water containing 4% trehalose, and freeze-dried for 48 h for later use. The average particle size of nanoparticles with target heads was about 270 nm, and about 80 μg of the protein and peptide components were loaded per 1 mg of PLGA nanoparticles, containing 0.04 mg of Poly(I:C) and CpGSL03 each. The control nanoparticles without adjuvants but with mannose target heads also had a particle size of about 270 nm, which were prepared using an equivalent quantity of cell components but without any immune adjuvant, and about 80 μg of the protein and peptide components were loaded per 1 mg of PLGA nanoparticles. The blank nanoparticles with mannose targets had a particle size of around 250 nm and were prepared using an equivalent quantity of the adjuvants, but not loaded with any cell lysis components.

### (3) Isolation and Expansion of Cancer Cell-Specific T Cells

Each C57BL/6 mouse was subcutaneously inoculated with 2.5×10⁵ B16F10 cells on the back on day 0, and the mice were subcutaneously injected with 100 μL of 1 mg PLGA nanoparticles on days 10, 15, and 20, respectively. On day 24, the mice were sacrificed and the tumor tissue and lymph nodes of the mice were harvested. The mouse tumor tissue and lymph nodes were respectively prepared into single-cell suspensions. CD45⁺ CD3⁺ T cells were then isolated from live cells (dead cells were labeled with live-dead cell dye to remove dead cells) in the tumor tissue single-cell suspension using flow cytometry. CD11c⁺ DC cells were isolated from live cells (dead cells were labeled with live-dead cell dye to remove dead cells) in splenocytes using flow cytometry. T cells (0.5 million cells), DC cells from lymph nodes (1 million cells) and DC2.4 cells (2 million cells) were co-incubated with nanoparticles loaded with the whole antigens of tumor tissue (100 μg) or control nanoparticles (100 μg) in DMEM high glucose medium for 72 hours (37° C., 5% CO₂), and then CD3⁺ CD69⁺ T cells, i.e., cancer cell-specific T cells, were sorted from the incubated cells by flow cytometry. The T cells obtained from the above sorting were co-incubated with IL-2 (2000 U/mL) and αCD-3 antibodies (50 ng/mL) in DMEM high glucose medium for 12 days (medium exchange every two days) to expand T cells.

### (4) Cancer Cell-Specific T Cells for Cancer Prevention

Female C57BL/6 mice aged 6-8 weeks were selected as model mice to prepare melanoma tumor-bearing mice. One day before adoptive transfer of cells, the recipient mice were intraperitoneally injected with a 100 mg/kg dose of cyclophosphamide to eliminate immune cells in the recipient mice. Then, 5 million cancer cell-specific T cells prepared in step (3) were intravenously injected into the recipient mice. Each recipient mouse was subcutaneously inoculated with 1.5×10⁵ B16F10 cells on the lower right side of the back the next day. The tumor growth rate of the mice and the survival of the mice were monitored. Tumor growth and survival monitoring methods were the same as above.

### (5) Experimental Results

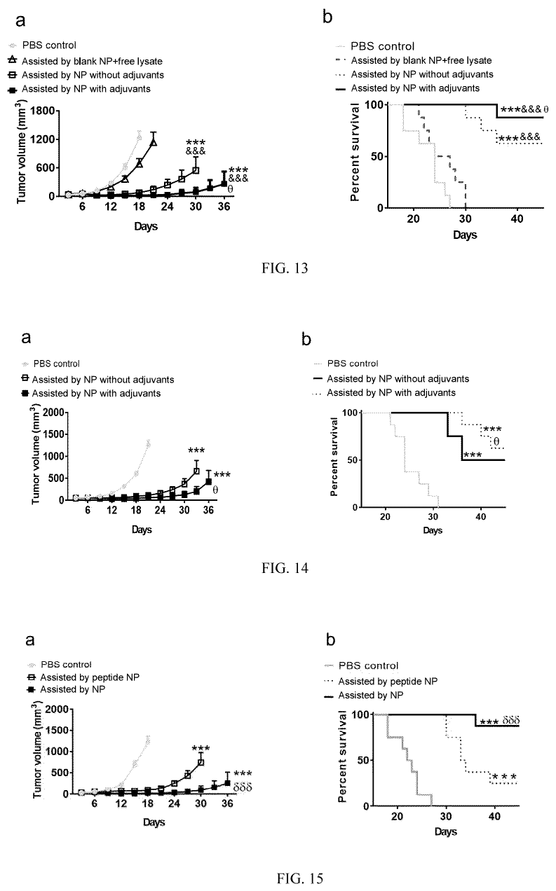

As shown in FIG. 13, the tumor growth rate of the mice treated with particle-assisted isolated cancer cell-specific T cells was significantly slower compared with the control group. Regardless of the adjuvants, nanoparticles can effectively assist in isolating cancer cell-specific T cells in tumor-infiltrating lymphocytes, but the effect was better with the adjuvants. This indicated that the cancer cell-specific T cells of the present disclosure can effectively prevent cancer.

### Example 13 Cancer Cell-Specific T Cells for Hepatocellular Carcinoma Prevention

In this example, first, Hepa1-6 hepatocellular carcinoma cells were lysed, PLGA was used as a nanoparticle skeleton material, and Poly(I:C) and BCG were used as immune adjuvants to prepare a nanoparticle system loaded with whole-cell antigens of cancer cells derived from hepatocellular carcinoma cells, then the particles were used to assist in isolating cancer cell-specific T cells derived from tumor-infiltrating lymphocytes, and the cells were isolated and extracted to prevent hepatocellular carcinoma.

### (1) Lysis and Component Collection of Cancer Cells

The cultured Hepa 1-6 hepatocellular carcinoma cells were collected and washed twice with PBS, and treated with heating and ultraviolet irradiation, and then an 8 M aqueous urea solution (containing 200 MM sodium chloride) was adopted to lyse and solubilize whole-cell antigens of cancer cells derived from cancer cells. The 8 M aqueous urea solution (containing 200 MM sodium chloride) was used to lyse BCG, and then the lysis components were solubilized to act as an adjuvant.

### (2) Preparation of a Nanoparticle System

In this example, the nanoparticle system was prepared by a solvent evaporation method, the molecular weight of the nanoparticle preparation material PLGA was 24 KDa to 38 KDa, the immune adjuvants were BCG and Poly(I:C), and the adjuvants were encapsulated in the nanoparticles. The preparation method was as previously described. In the preparation process, the whole-cell antigens of cancer cells and adjuvants were first loaded inside the nanoparticles by the double-emulsion method, and after the antigens (lysis components) were loaded inside, 100 mg nanoparticles were centrifuged at 10000 g for 20 minutes and freeze-dried for 48 h after resuspension using 10 mL of ultrapure water containing 4% trehalose for later use. The average particle size of the nanoparticles was about 270 nm; and approximately 100 μg of the protein and peptide components were loaded per 1 mg of PLGA nanoparticles, with 0.04 mg of BCG and Poly(I:C) each. The average particle size of the control nanoparticles was about 270 nm, and each 1 mg of PLGA nanoparticles was loaded with about 100 μg of the protein and peptide components, without any adjuvant.

### (3) Isolation and Expansion of Cancer Cell-Specific T Cells

Each C57BL/6 mouse was subcutaneously inoculated with 2×10⁶ Hepa 1-6 cells on the back on day 0, and the mice were subcutaneously injected with 1 mg of PLGA nanoparticles on days 10, 14, 21, and 28, respectively. The mice were sacrificed on day 33, the tumor tissue and lymph nodes of the mice were harvested, and single-cell suspensions of the tumor tissue and lymph nodes were prepared. CD45⁺ CD3⁺ T cells were isolated from live cells (dead cells were labeled with live-dead cell dye to remove dead cells) in the tumor tissue single-cell suspension using flow cytometry. CD19⁺ B cells and CD11c⁺ DC cells were isolated from viable cells (dead cells were labeled with live-dead cell dye to remove dead cells) in the mouse lymph node single-cell suspension. The isolated T cells (0.4 million cells), B cells (2 million cells) and DC cells (2 million cells) were co-incubated with nanoparticles loaded with the whole antigens of tumor tissue (100 μg) or control nanoparticles (100 μg) in DMEM high glucose medium for 48 hours (37° C., 5% CO₂), and then CD3⁺ CD69⁺ T cells, i.e., cancer cell-specific T cells, were isolated from the incubated cells by flow cytometry. The cancer cell-specific T cells obtained by the above sorting were co-incubated with IL-2 (1000 U/mL) and αCD-3 antibodies (60 ng/mL) in DMEM high glucose medium for 14 days (medium change every two days) to expand T cells.

### (4) Prevention of Cancer Cell-Specific Cancer

Female C57BL/6 at 6-8 weeks were selected as model mice to prepare hepatocellular carcinoma tumor-bearing mice. One day before adoptive transfer of cells, the recipient mice were intraperitoneally injected with a 100 mg/kg dose of cyclophosphamide to eliminate immune cells in the recipient mice. The mice were injected with 4 million cancer cell-specific T cells on day 0. At the same time, each mouse was inoculated with 1.0×10⁶ Hepa1-6 hepatocellular carcinoma cells subcutaneously on day 0, and the tumor growth and survival of the mice were recorded in the same manner as above.

### (5) Experimental Results

As shown in FIG. 14, the tumor growth rate of the mice treated with nanoparticle-assisted isolated cancer cell-specific T cells was significantly slower compared with the control group. Moreover, regardless of the adjuvants, nanoparticles can effectively activate and assist in isolating cancer cell-specific T cells in tumor-infiltrating lymphocytes, but the effect was better with the adjuvants. This indicated that the T cells of the present disclosure can effectively prevent cancer.

### Example 14 Cancer Cell-Specific T Cells Isolated with Assistance of Calcified Nanoparticles for Cancer Prevention

This example illustrated that calcified nanoparticles assist in isolating cancer cell-specific T cells in tumor-infiltrating lymphocytes, and other biomineralization techniques, crosslinking, gelation, etc. can also be used to modify the particles in actual use. In this example, mouse melanoma tumor tissue and cancer cells were lysed with 8 M urea and then lysed, and then the tumor tissue lysis components and the cancer cell lysis components were loaded on a nanoparticle system at a mass ratio of 1:1, and the particles were used to assist in isolating cancer cell-specific T cells in tumor tissue-infiltrating lymphocytes to prevent cancer after expansion of the cancer cell-specific T cells. In this example, nanoparticles loaded with four peptide neoantigens B16-M20 (Tubb3, FRRKAFLHWYTGEAMDEMEFTEAESNM) (SEQ ID NO: 1), B16-M24 (Dag1, TAVITPPTTTTKKARVSTPKPATPSTD) (SEQ ID NO: 2), B16-M46 (Actn4, NHSGLVTFQAFIDVMSRETTDTDTADQ) (SEQ ID NO: 3) and TRP2:180-188(SVYDFFVWL) (SEQ ID NO: 4) were used as control nanoparticles.

### (1) Lysis of Tumor Tissue and Cancer Cells

After collecting mouse B16F10 melanoma tumor tissue and cultured cancer cells, whole-cell antigens of cancer cells derived from the tumor tissue and cancer cells were lysed and solubilized with 8 M urea, and then the tumor tissue components and the cancer cell components were mixed at a mass ratio of 1:1.

### (2) Preparation of Nanoparticles

This example biocalcified the nanoparticles after loading whole-cell antigens of cancer cells inside and on the surface of the nanoparticles. In this example, the nanoparticles were prepared by a solvent evaporation method, the molecular weight of the nanoparticle preparation material PLGA adopted was 7 KDa to 17 KDa, and the immune adjuvants CpG2006 and Poly(I:C) adopted were loaded inside the nanoparticles. The preparation method was as follows. In the preparation process, the antigens were first loaded inside the nanoparticles by a double-emulsion method. After the lysis components were loaded inside, 100 mg of PLGA nanoparticles were centrifuged at 13000 g for 20 minutes and then resuspended with 18 mL of PBS. Then, 2 mL of the tumor tissue and cancer cell lysate (60 mg/mL) solubilized in 8 M urea was added, and the precipitate was collected after centrifugation at 12000 g for 20 minutes after 10 minutes at room temperature. The 100 mg PLGA nanoparticles were then resuspended in 20 mL of DMEM medium, and then 200 μL of CaCl₂ (1 mM) was added and reacted at 37° C. for two hours. The precipitate was then collected after centrifugation at 10000 g for 20 minutes and resuspended with ultrapure water and centrifuged twice. The average particle size of the nanoparticles was about 290 nm; and approximately 140 μg of the protein or peptide components were loaded per 1 mg of PLGA nanoparticles, with 0.03 mg of CpG2006 and Poly(I:C) each. The preparation method of control nanoparticles loaded with various antigen peptides was the same as above. The particle size of the control nanoparticles was about 290 nm, and about 140 μg of antigen peptides and an equivalent quantity of the adjuvants were loaded per 1 mg of PLGA nanoparticles.

### (3) Isolation and Expansion of Cancer Cell-Specific T Cells

Same as Example 13.

### (4) Cancer Cell-Specific T Cells for Cancer Prevention

Same as Example 1.

### (5) Experimental Results

As shown in FIG. 15, compared with the control group, the cancer cell-specific T cells obtained by isolation and expansion with assistance of calcified nanoparticles can prolong the survival time of the mice and effectively prevent cancer. Moreover, the cancer cell-specific T cells obtained by isolation and expansion with assistance of the nanoparticles loaded with the whole-cell antigens of cancer cells were better than the cancer cell-specific T cells obtained by isolation and expansion assisted by the nanoparticles loaded with the four antigen peptides.

### Example 15 Cancer Cell-Specific T Cells for Melanoma Treatment

In this example, mouse melanoma was used as a cancer model to illustrate how nanoparticles were used to activate and assist in isolating cancer cell-specific T cells in tumor-infiltrating lymphocytes, and the above cells were expanded and infused back into mice to treat melanoma.

### (1) Lysis and Component Collection of Tumor Tissue and Cancer Cells

When collecting tumor tissue, 1.5×10⁵ B16F10 cells were subcutaneously inoculated on the back of each C57BL/6 mouse. When the tumor grew to about 1000 mm³, the mice were sacrificed and the tumor tissue was removed. The tumor tissue was cut into pieces and ground, and a single-cell suspension was prepared after passing through a cell strainer. Freezing and thawing was repeated after adding ultrapure water, accompanied by ultrasound to lyse the above cells, and then nuclease was added for reaction for 5 minutes and then inactivated at 95° C. for 10 minutes. Then the cells were centrifuged at 8000 g for 3 minutes, and the supernatant part was water-soluble antigens; and in the precipitated part, water-insoluble antigens were solubilized using a 10% aqueous sodium deoxycholate solution. The water-soluble antigens and the water-insoluble antigens solubilized by sodium deoxycholate were mixed at a mass ratio of 1:1 to obtain the source of antigen raw materials for preparing the nanoparticle system.

### (2) Preparation of a Nanoparticle System

In this example, the nanoparticles were prepared by a double-emulsion method and had the ability to target dendritic cells. The nanoparticle preparation materials adopted were PLGA and mannan-modified PLGA, both of which had molecular weights of 24 KDa to 38 KDa, and the mass ratio of unmodified PLGA and mannan-modified PLGA was 9:1 when used. The immune adjuvants adopted were poly(I:C), CpG1018 and CpG2216, and the substance that increased lysosome immune escape was KALA peptide (WEAKLAKALAKALAKHLAKALAKALKACEA)(SEQ ID NO: 5), and the adjuvant and KALA peptide were encapsulated in the nanoparticles. The preparation method was as previously described. In the preparation process, the lysate components, adjuvant and KALA peptide were first loaded inside the nanoparticles by the double-emulsion method. After the above components were loaded inside, 100 mg nanoparticles were centrifuged at 12000 g for 25 minutes and freeze-dried for 48 h after resuspension using 10 mL of ultrapure water containing 4% trehalose. The average particle size of the nanoparticles was about 250 nm, and the surface potential was about −5 mV; and approximately 100 μg of the protein or peptide components were loaded per 1 mg of PLGA nanoparticles, and 0.02 mg of each of poly(I:C), CpG1018 and CpG2216 immune adjuvants, and 0.05 mg of KALA peptide were loaded per 1 mg of PLGA nanoparticles. The preparation materials and method of Nanoparticles 2 were the same. The particle size of Nanoparticles 2 was about 250 nm, the surface potential was about −5 mV, it was not loaded with KALA peptide, and it was loaded with an equivalent quantity of the adjuvants and cell lysis components. The preparation materials and preparation method of Nanoparticles 3 were the same. The Nanoparticles 3 was about 250 nm, and the surface potential was about −5 mV; and about 100 μg of the protein and peptide components were loaded per 1 mg of PLGA nanoparticles, and 0.02 mg of poly(I:C), 0.04 mg of CpG1018, and 0.05 mg of KALA peptide were loaded per 1 mg of PLGA nanoparticles.

### (3) Preparation of Cancer Cell-Specific T Cells

Female C57BL/6 mice aged 6-8 weeks were subcutaneously inoculated with 1.5×10⁵ B16F10 on the back of the mice on day 0, and then the mice were subcutaneously injected with 0.5 mg of PLGA nanoparticles (loaded with lysate components, Poly(I:C) and two CpG adjuvants and KALA peptide) on days 15, 20 and 25, respectively. The mice were sacrificed on day 30, and the tumor mass and lymph nodes of the mice were collected. A single-cell suspension was prepared through a cell strainer after cutting the mouse tumor mass into small pieces, and then T cells in CD45⁺ CD3⁺ tumor-infiltrating lymphocytes were sorted from live cells (dead cells were labeled with live-dead cell dye to remove dead cells) in these cells using magnetic bead sorting. CD11c⁺ DC cells and CD19′ B cells were isolated from live cells (dead cells were labeled with live-dead cell dye to remove dead cells) in the lymph node single-cell suspension. The sorted CD3⁺ T cells (0.5 million cells), nanoparticles (40 μg), DC cells from lymph nodes (1 million cells), B cells (1 million cells), and IL-7 (10 ng/mL) were co-incubated in 2 mL of RPMI 1640 complete medium for 96 hours. CD3⁺OX40⁺ cells, i.e., cancer cell-specific T cells that can recognize whole-cell antigens of cancer cells, are then sorted from the incubated T cells by flow cytometry. CD3⁺OX40⁺ cells obtained by the above sorting were co-incubated with IL-2 (1000 U/mL), IL-15 (1000 U/mL), IL-21 (1000 U/mL), and αCD-3 antibodies (20 ng/mL) in RPMI 1640 complete medium for 14 days (medium change every two days) to expand cancer cell-specific T cells.

### (4) Expanded Cancer Cell-Specific T Cells for Cancer Treatment

Female C57BL/6 aged 6-8 weeks were selected as model mice to prepare melanoma-bearing mice. Each mouse was subcutaneously inoculated with 1.5×10⁵ B16F10 cells on the lower right side of the back on day 0. 1.5 million expanded cancer cell-specific T cells were intravenously injected on days 4, 7, 10, 15 and 20 respectively after melanoma inoculation. In the experiment, the tumor volume and survival of the mice were monitored by the same method as above.

### (5) Experimental Results

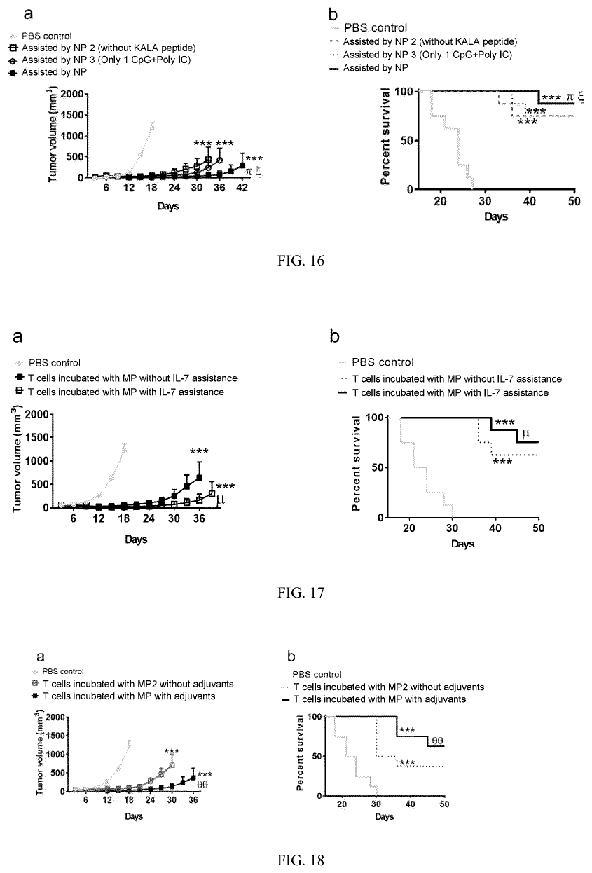

As shown in FIG. 16, the tumors in the PBS control group all grew. Compared with the control group, the tumor growth rate of the mice treated with cancer cell-specific T cells was significantly slower and the survival time was significantly prolonged. Moreover, cancer cell-specific T cells isolated and expanded with assistance of adding nanoparticles loaded with substances that increased lysosome escape were better than cancer cell-specific T cells isolated and expanded with assistance of adding nanoparticles without substances that increased lysosome escape; and the therapeutic effect of cancer cell-specific T cells isolated and expanded with assistance of using two CpG and Poly(I:C) as a mixed adjuvant was better than that using only one of the CpG and Poly(I:C) mixed adjuvant. In summary, the cancer cell-specific T cells of the present disclosure had a good therapeutic effect on cancer.

### Example 16 Cancer Cell-Specific T Cells for Breast Cancer Prevention

This example used 4T1 mouse triple-negative breast cancer as a cancer model to illustrate how microparticles loaded with whole-cell antigens of cancer cells were used to assist in isolating cancer cell-specific T cells for the prevention of breast cancer. In this example, breast cancer cells were first subjected to inactivation and denaturation treatment, then the cells were lysed, and water-insoluble antigens in the lysed cancer cells were solubilized with octyl glucoside. Then, a microparticle system loaded with whole-cell antigens of cancer cells was prepared by using PLGA as a microparticle backbone material, CpG2007, CpG1018, and Poly ICLC as immune adjuvants, and polyarginine and polylysine as substances to enhance lysosome escape.

### (1) Lysis of Cancer Cells

The cultured 4T1 cells were centrifuged at 400 g for 5 minutes, then washed twice with PBS and resuspended in ultrapure water. The obtained cancer cells were inactivated and denatured by ultraviolet and high-temperature heating respectively, then ultrapure water was added and freezing-thawing cycle was repeated 5 times, accompanied by ultrasound to lyse the cancer cells, the cell lysate was centrifuged at 5000 g for 10 minutes, the supernatant was the water-soluble antigen, the precipitate was solubilized by using 10% octyl glucoside to obtain solubilized original water-insoluble antigens, and the water-soluble antigens and the water-insoluble antigens were mixed at a mass ratio of 2:1 to obtain the lysate components required for preparing the microparticles.

### (2) Preparation of a Microparticle System

In this example, the microparticle system and the control microparticles were prepared by a double-emulsion method, the microparticle skeleton material PLGA had a molecular weight of 38 KDa to 54 KDa, the immune adjuvants adopted were CpG2007, CpG1018 and Poly ICLC, and the lysosome escape-increasing substances adopted were polyarginine and polylysine. During preparation, the microparticles internally loaded with the lysate components, adjuvants and KALA peptide were prepared by the double-emulsion method. After the lysate and adjuvants were internally loaded, 100 mg of the microparticles were centrifuged at 9000 g for 20 minutes, and resuspended using 10 mL of ultrapure water containing 4% trehalose and then dried for 48 h for later use. The average particle size of the microparticle system was about 3.1 μm, and the surface potential of the microparticle system was about −7 mV; and each 1 mg of PLGA microparticles were loaded with about 110 μg of the protein or peptide components, 0.01 mg each of CpG and Poly ICLC, and 0.02 mg each of polyarginine and polylysine.

### (3) Preparation of Dendritic Cells

This example illustrated how to prepare bone marrow-derived dendritic cells (BMDCs) by taking the preparation of dendritic cells from mouse bone marrow cells as an example. First, a 6-8-week-old C57 mouse was sacrificed by cervical dislocation. The tibia and femur of the hind leg were surgically removed and put into PBS, and the muscle tissue around the bone was removed cleanly with scissors and tweezers. Both ends of the bone were cut off with scissors, then a PBS solution was extracted with a syringe, needles were inserted from both ends of the bone into the bone marrow cavity, and the bone marrow was repeatedly rinsed into a culture dish. The bone marrow solution was collected, centrifuged at 400 g for 3 min, and then added with 1 mL of a red blood cell lysate to lyse red blood cells. 3 mL of RPMI 1640 (10% FBS) medium was added to terminate lysis, centrifugation was performed at 400 g for 3 min, and the supernatant was discarded. Cells were cultured in a 10 mm culture dish using RPMI 1640 (10% FBS) medium with the addition of recombinant mouse GM-CSF (20 ng/mL) at 37° C. and 5% CO₂ for 7 days. On Day 3, the culture flask was gently shaken and the same volume of RPMI 1640 (10% FBS) medium containing GM-CSF (20 ng/mL) was replenished. On day 6, the medium was subjected to a half-volume change treatment. On day 7, a small number of suspended and semi-adherent cells were collected. When the ratio of CD86⁺ CD80⁺ cells in CD11c⁺ cells was between 15-20% by flow cytometry, the induced cultured BMDC could be used for the next experiment.

### (4) Preparation of Cancer Cell-Specific T Cells

Female BALB/c mice aged 6-8 weeks were selected, and 2×10⁶ 4T1 breast cancer cells were subcutaneously inoculated on the back of the mice on day 0; and the microparticles of 0.3 mg of PLGA (loaded with the lysate components, adjuvants and substances increasing lysosome escape) were injected subcutaneously on days 7, 14, 21, and 28, respectively. The mice were sacrificed on day 32, the tumor tissue of the mice was collected, and the tumor tissue was cut into small pieces and passed through a cell strainer to prepare a single-cell suspension. CD45⁺ tumor-infiltrating lymphocytes were sorted from live cells (dead cells were labeled with live-dead cell dye to remove dead cells) in the tumor tissue single-cell suspension using flow cytometry. The sorted CD45⁺ cells (1 million cells), microparticles (100 μg, loaded with the lysate part, adjuvants and substances increasing lysosome escape), BMDC (2 million cells), and IL-7 (10 ng/mL) were co-incubated in 5 mL of RPMI 1640 complete medium for 48 hours (37° C., 5% CO₂); and alternatively, the sorted CD45⁺ cells (1 million cells), microparticles (100 μg, loaded with the lysate part, adjuvants and substances increasing lysosome escape) and BMDC (2 million cells) were co-incubated in 5 mL of RPMI 1640 complete medium for 48 hours (37° C., 5% CO₂). Then, CD3⁺ CD8⁺ CD69⁺ T cells and CD3⁺ CD4⁺ CD69⁺ T cells, i.e., cancer cell-specific T cells that can recognize whole-cell antigens of cancer cells, were sorted from the incubated CD45⁺ T cells by flow cytometry. The sorted CD8⁺ CD69⁺ T cells or CD4⁺ CD69⁺ T cells obtained by the above sorting were co-incubated with IL-2 (1000 U/mL), IL-6 (1000 U/mL), IL-12 (1000 U/mL), and αCD-28 antibodies (10 ng/mL) in RPMI 1640 complete medium for 14 days to expand cancer cell-specific T cells.

### (5) Cancer Cell-Specific T Cells for Cancer Prevention

Female BALB/c at 6-8 weeks were selected as model mice to prepare breast cancer tumor-bearing mice. One day before the adoptive transfer of cells, the recipient mice were intraperitoneally injected with a 100 mg/kg dose of cyclophosphamide to eliminate immune cells in the recipient mice. The mice were subcutaneously injected with 100 μL containing 0.6 million expanded CD8⁺ T cells and 0.4 million expanded CD4⁺ T cells on day 0. At the same time, each mouse was inoculated with 1×10⁶ 4T1 cells subcutaneously on day 0, and the tumor volume size of the mice was recorded every 3 days from day 3. The tumor volume was calculated using the formula v=0.52×a×b², where v was the tumor volume, a was the tumor length, and b was the tumor width. Due to animal experimental ethics, when the tumor volume of the mouse exceeded 2000 mm³ in the mouse survival test, the mouse was regarded as dead and the mouse was euthanized.

### (6) Experimental Results

As shown in FIG. 17, compared with the control group, the tumor growth rate of the cancer cell-specific T cell treatment group obtained by microparticle-assisted sorting was significantly slower and the survival time of the mice was significantly prolonged. Moreover, the effect of cancer cell-specific T cells obtained by adding IL-7 to assist in sorting during co-incubation was better than that of cancer cell-specific T cells obtained without adding IL-7 to assist in sorting during co-incubation. It can be seen that the cancer cell-specific T cells of the present disclosure had a preventive effect on breast cancer.

### Example 17 Cancer Cell-Specific T Cells for Breast Cancer Prevention

This example used 4T1 mouse triple-negative breast cancer as a cancer model to illustrate how a microparticle system assisted in sorting cancer cell-specific T cells, and the T cells were used to prevent breast cancer after expansion.

### (1) Lysis of Cancer Cells

The cultured 4T1 cells were centrifuged at 400 g for 5 minutes, then washed twice with PBS and resuspended in ultrapure water. The obtained cancer cells were inactivated and denatured by ultraviolet rays and high-temperature heating, respectively, and then the cancer cells were lysed and the lysate components were solubilized by 8 M aqueous urea solution (containing 500 mM sodium chloride), which was the antigen components for preparing the microparticle system.

### (2) Preparation of a Microparticle System

In this example, the microparticle system and the control microparticles were prepared by a double-emulsion method, the microparticle skeleton materials were unmodified PLA and mannose-modified PLA, both of which had a molecular weight of 40 KDa, and the ratio of unmodified PLA and mannose-modified PLA was 4:1. The immune adjuvants adopted were CpG2006, CpG2216 and Poly ICLC, and the lysosome escape-increasing substances adopted were arginine and histidine. During preparation, the microparticles internally loaded with the lysate components, adjuvants, arginine and histidine were prepared by the double-emulsion method. Then, 100 mg of the microparticles were centrifuged at 9000 g for 20 minutes, and resuspended using 10 mL of ultrapure water containing 4% trehalose and then dried for 48 h for later use. The average particle size of the microparticle system was about 2.1 μm, and the surface potential of the microparticle system was about −7 mV; and each 1 mg of PLGA microparticles was loaded with about 100 μg of the protein or peptide components, containing 0.01 mg of CpG2006, CpG2216 and Poly ICLC each, and containing 0.05 mg of arginine and histidine each. The preparation materials and preparation method of control Microparticles 2 were the same as those described in this example. Microparticles 2 had a particle size of about 2.1 μm, and a surface potential of about −7 mV, and were loaded with only arginine and histidine and an equivalent quantity of cell lysate components without any adjuvant.

### (3) Preparation of Cancer Cell-Specific T Cells

Female BALB/c mice aged 6-8 weeks were selected, and 2×10⁶ 4T1 breast cancer cells were inoculated subcutaneously on the back of the mice on day 0. 100 μL of the microparticles containing 0.2 mg of PLGA (loaded with the lysate components, adjuvants and substances increasing lysosome escape) were injected subcutaneously on days 7, 14, 21, and 28, respectively. The mice were sacrificed on day 32, the tumor tissue of the mice was collected, then tumor tissue single-cell suspensions were prepared, and CD3⁺ T cells were sorted from live cells (dead cells were labeled with live-dead cell dye to remove dead cells) in the tumor tissue single-cell suspension using flow cytometry. The sorted CD3⁺ T T cells (0.2 million cells), microparticles (50 μg), DC2.4 cells (0.5 million cells), and IL-7 (10 ng/mL) were co-incubated in 2 mL of RPMI 1640 complete medium for 48 hours (37° C., 5% CO₂), and then CD3⁺ CD8⁺ CD69⁺ T cells were sorted from the incubated CD3⁺ T cells and CD4⁺ CD69⁺ T cells were sorted from the CD4⁺ T cells by flow cytometry, where the CD3⁺ CD8⁺ CD69⁺ T cells and CD4⁺CD69⁺ T cells were cancer cell-specific T cells that can recognize whole-cell antigens of cancer cells. The sorted CD8⁺ CD69⁺ T cells or CD4⁺ CD69⁺ T cells were co-incubated with IL-2 (2000 U/mL), IL-7 (1000 U/mL) and αCD-3 antibodies (10 ng/mL) in RPMI 1640 complete medium for 14 days to expand cancer cell-specific T cells.

### (4) Cancer Cell-Specific T Cells for Cancer Prevention

Female BALB/c at 6-8 weeks were selected as model mice to prepare breast cancer tumor-bearing mice. One day before the adoptive transfer of cells, the recipient mice were intraperitoneally injected with a 100 mg/kg dose of cyclophosphamide to eliminate immune cells in the recipient mice. The mice were subcutaneously injected with 1 million expanded CD8⁺ T cells and 0.4 million expanded CD4⁺ T cells on day 0. At the same time, each mouse was inoculated with 1×10⁶ 4T1 cells subcutaneously on day 0, and the tumor volume and survival of the mice were monitored as above.

### (5) Experimental Results

As shown in FIG. 18, the mice treated with expanded cancer cell-specific T cells obtained by activation and assisted isolation using microparticles had a significantly slower tumor growth rate and significantly prolonged mouse survival time compared with the control group. Furthermore, the cancer cell-specific T cells isolated by the microparticles containing the substance increasing the lysosome escape function and the mixed adjuvant were more effective than the cancer cell-specific T cells isolated by the microparticles containing only the substance increasing the lysosome escape function and not containing the mixed adjuvant. Therefore, it can be seen that the cancer cell-specific T cells of the present disclosure had a preventive effect on breast cancer, and the use of the mixed adjuvant facilitated the isolation and expansion of the cancer cell-specific T cells.

### Example 18 Cancer Cell-Specific T Cells for Melanoma Treatment

This example used mouse melanoma as a cancer model to illustrate how nanoparticles were used to assist in sorting cancer cell-specific T cells, and the T cells were expanded and infused back to treat melanoma. In this example, first, tumor tissue and cancer cells are lysed to prepare water-soluble antigens and water-insoluble antigens, then, PLGA was used as a backbone material, Poly(I:C) and CpG as immune adjuvants, and R8 (RRRRRRRR)(SEQ ID NO: 6) peptide as a substance having the lysosome escape function to prepare a nanoparticle system loaded with the water-soluble antigens or the water-insoluble antigens, then the nanoparticles were co-incubated with dendritic cells and T cells in vitro, and then activated cancer cell-specific T cells were sorted and then expanded and infused back to treat cancer.

### (1) Lysis and Component Collection of Tumor Tissue and Cancer Cells

When collecting tumor tissue, 1.5×10⁵ B16F10 cells were subcutaneously inoculated on the back of each C57BL/6 mouse. When the tumor grew to about 1000 mm³, the mice were sacrificed and the tumor tissue was removed. The tumor tissue was cut into pieces and ground, and the sample was filtered through the cell strainer and an appropriate amount of pure water was added, and frozing and thawing was repeated 5 times (with ultrasound) to destroy and lyse the obtained sample. After adding nuclease for 10 minutes, the nuclease was inactivated by heating at 95° C. for 10 minutes. When collecting the cultured B16F10 cancer cell line, the culture medium was removed by centrifugation, then the cancer cells were washed twice with PBS, collected by centrifugation, resuspended in ultrapure water, and repeatedly frozen and thawed 3 times, accompanied by ultrasound to destroy and lyse the cancer cells, and then nuclease was added to the sample for 10 minutes and then heated at 95° C. for 5 minutes to inactivate the nuclease. After the tumor tissue or cancer cells were treated by enzyme action, the lysate was centrifuged at a rotational speed of 5000 g for 5 minutes and the supernatant was taken to obtain water-soluble antigens soluble in pure water; and the obtained precipitated part was solubilized by adding an 8 M aqueous urea solution to the precipitated part. The water-soluble antigens of the tumor tissue and the water-soluble antigens of the cancer cells were mixed at a mass ratio of 1:1; and the water-insoluble antigens of the tumor tissue and the water-insoluble antigens of the cancer cells were mixed at a mass ratio of 1:1. Then the water-soluble antigen mixture and the water-insoluble antigen mixture were mixed at a mass ratio of 2:1, which was the source of antigen raw materials for preparing the nanoparticle system.

### (2) Preparation of a Nanoparticle System

The nanoparticles in this example were prepared by a double-emulsion method. The molecular weight of the nanoparticle preparation material PLGA adopted was 7 KDa to 17 KDa, the immune adjuvants adopted were poly(I:C) and CpG1018, the R8 peptide was a substance that increased lysosome escape, and the adjuvants and the R8 peptide were loaded inside the nanoparticles. The preparation method was as previously described. In the preparation process, the lysate components, adjuvants and R8 peptide were first loaded inside the nanoparticles by the double-emulsion method. After completion of the internal loading, 100 mg nanoparticles were centrifuged at 12000 g for 25 minutes, and resuspended with 10 mL of ultrapure water containing 4% trehalose and then freeze-dried for 48 h; and before use, the nanoparticles were resuspended in 9 mL of PBS and then 1 mL of the lysate components (protein concentration of 80 mg/mL) was added for reaction at room temperature for 10 min to obtain a nanoparticle system loaded with the lysate both inside and outside. The average particle size of the nanoparticles was about 290 nm, and the surface potential of the nanoparticles was about −5 mV; and approximately 140 μg of the protein or peptide components were loaded per 1 mg of PLGA nanoparticles, 0.02 mg of each of poly(I:C) and CpG1018 immune adjuvants and 0.01 mg of R8 peptide were loaded per 1 mg of PLGA nanoparticles.

### (3) Preparation of Cancer Cell-Specific T Cells

Female C57BL/6 mice aged 6-8 weeks were subcutaneously inoculated with 8×10⁵ B16F10 cells on the back on day 0, and the mice were subcutaneously injected with 100 L of 0.5 mg PLGA nanoparticles on days 7, 14, 21, and 28, respectively. The mice were sacrificed on day 32, and the tumor tissue and splenocytes of the mice were collected. A single-cell suspension of the mouse tumor tissue and a single-cell suspension of the splenocytes were prepared. Then CD45⁺CD3⁺ T cells were isolated from live cells (dead cells were labeled with live-dead cell dye to remove dead cells) in the mouse tumor tissue single-cell suspension and CD19⁺ B cells were isolated from live cells (dead cells were labeled with live-dead cell dye to remove dead cells) in the splenocyte single-cell suspension using flow cytometry. The sorted CD3⁺ T cells (1 million cells), nanoparticles (100 μg), DC2.4 cell line (3 million cells), B cells (2 million cells), and IL-7 (10 ng/mL) were co-incubated in 5 mL of RPMI 1640 complete medium for 72 hours, and then the incubated CD3⁺ T cells were sorted by flow cytometry for CD3⁺ OX40⁺ T cells, i.e., cancer cell-specific T cells that can recognize whole-cell antigens of cancer cells. The cancer cell-specific T cells obtained by the above sorting were co-incubated with IL-2 (2000 U/mL) and αCD-3 antibodies (10 ng/mL) in RPMI 1640 complete medium for 14 days (medium change every two days) to expand the cancer cell-specific T cells.

As a control, CD3⁺ T cells isolated from live cells in the single-cell suspension of mouse tumor tissue (dead cells were labeled with live-dead cell dye to remove dead cells) were directly screened for CD3⁺ OX40⁺ T cells by flow cytometry without co-incubation with nanoparticles and antigen-presenting cells, and directly co-incubated with IL-2 (2000 U/mL) and αCD-3 antibodies (10 ng/mL) in RPMI 1640 complete medium for 14 days (medium change every two days) to expand the cancer cell-specific T cells.

### (4) Administration of Allogeneic Cell Mixtures to Cancer-Bearing Mice for Cancer Treatment

Female C57BL/6 aged 6-8 weeks were selected as model mice to prepare melanoma-bearing mice. Each mouse was subcutaneously inoculated with 1.5×10⁵ B16F10 cells on the lower right side of the back on day 0. 1.5 million expanded cancer-specific CD3⁺ T cells were intravenously injected on days 4, 7, 10, 15, and 20 respectively after melanoma inoculation. The methods of monitoring tumor growth and survival in the mice were the same as above.

### (5) Experimental Results

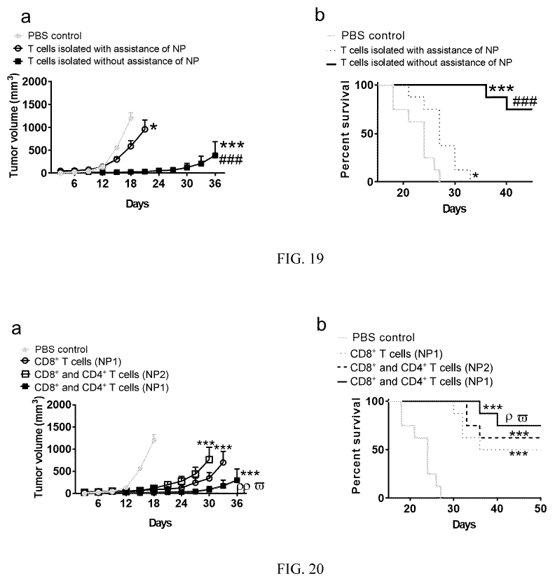

As shown in FIG. 19, the tumor growth rate of the mice treated with expanded cancer cell-specific T cells was significantly slower and the survival time of the mice was significantly prolonged compared with the control group. Moreover, cancer cell-specific CD3⁺ T cells obtained using nanoparticle-assisted isolation were better than CD3⁺ T cells directly expanded without nanoparticle-assisted isolation. Therefore, it can be seen that the cancer cell-specific T cells of the present disclosure had a preventive effect on cancer, and the nanoparticle-assisted isolation had a significant enhancement effect.

### Example 19 Cancer Cell-Specific T Cells for Colon Cancer Treatment

This example used mouse colon cancer as a cancer model to illustrate how nanoparticles loaded with whole-cell antigens of cancer cells derived from colon cancer tumor tissue were used to assist in sorting cancer cell-specific T cells and to treat colon cancer. In this example, first, an 8 M aqueous urea solution was used to lyse colon cancer tumor tissue and solubilize the lysed components, then, PLGA was used as a backbone material, Poly(I:C), CpG2336 and CpG2006 were used as adjuvants, and NH₄HCO₃ was used as a substance increasing lysosome escape to prepare a nanoparticle system, and then the cancer cell-specific T cells were used to assist in sorting, and the cancer cell-specific T cells obtained after two steps of sorting were expanded and used for cancer treatment.

### (1) Lysis and Component Collection of Tumor Tissue

When collecting tumor tissue, 2×10⁶ MC38 colon cancer cells were subcutaneously inoculated on the back of each C57BL/6 mouse. When the tumor grew to a volume of about 1000 mm³, the mice were sacrificed and the tumor tissue was removed. The tumor tissue was cut into pieces and ground. The tumor tissue was lysed by adding the 8 M aqueous urea solution through the cell strainer and the lysed components were solubilized. The above was the source of antigen raw materials for preparing the nanoparticle system.

### (2) Preparation of a Nanoparticle System

The nanoparticles in this example were prepared by a double-emulsion method. The molecular weight of the preparation material PLGA of Nanoparticles 1 was 7 KDa to 17 KDa, Poly(I:C) and CpG were used as adjuvants, and NH₄HCO₃ was used as a substance to increase lysosome escape, and the adjuvant and NH₄HCO₃ were loaded inside the nanoparticles; and the preparation method was as described previously. In the preparation process, the lysate components and adjuvants were first loaded inside the nanoparticles, and then 100 mg nanoparticles were centrifuged at 10000 g for 20 minutes, and resuspended with 10 mL of ultrapure water containing 4% trehalose and then freeze-dried for 48 h for later use; the average particle size of the nanoparticles was about 260 nm, and the surface potential was about −7 mV; and about 90 μg of the protein and peptide components were loaded per 1 mg of PLGA nanoparticles, and 0.02 mg each of poly(I:C), CpG2336 and CpG2006 immune adjuvants, and 0.01 mg of NH₄HCO₃ were loaded per 1 mg of PLGA nanoparticles. The preparation materials and preparation method of Nanoparticles 2 were the same as those of Nanoparticles 1. The particle size was about 260 nm, the surface potential was about −7 mV, about 90 μg of the protein and peptide components were loaded per 1 mg of PLGA nanoparticles, and 0.01 mg of NH₄HCO₃, and 0.03 mg each of CpG2336 and CpG2006 were loaded per 1 mg of PLGA nanoparticles.

### (3) Preparation of Cancer Cell-Specific T Cells

Female C57BL/6 mice aged 6-8 weeks were subcutaneously inoculated with 2×10⁶ MC38 colon cancer cells on the back on Day 0, and the mice were subcutaneously injected with 100 L nanoparticles containing 0.4 mg PLGA (loaded with the lysate part, mixed adjuvants and substances increasing lysosome escape) on Day 14 and Day 28, respectively. The mice were sacrificed on day 32, the tumor tissue of the mice was harvested and tumor tissue single-cell suspensions were prepared, and then CD8⁺ T cells and CD4⁺ T cells were sorted from live cells (dead cells were labeled with live-dead cell dye to remove dead cells) in the tumor tissue single-cell suspensions using flow cytometry. The sorted CD8⁺ T cells (0.2 million cells), CD4⁺ T cells (0.1 million cells), nanoparticles (50 μg), B cells (1 million cells), and IL-7 (10 ng/mL) were co-incubated in 2 mL of RPMI 1640 complete medium for 48 hours (37° C., 5% CO₂), and then CD8⁺ CD69⁺ T cells in the incubated CD8⁺ T cells and CD4⁺ CD69⁺ T cells in the CD4⁺ T cells were sorted by flow cytometry, where the CD8⁺ CD69⁺ T cells and CD4⁺ CD69⁺ T cells were cancer cell-specific T cells that can recognize whole-cell antigens of cancer cells. The sorted CD8⁺ CD69⁺ T cells or CD4⁺ CD69⁺ T cells obtained by the above sorting were co-incubated with IL-2 (1000 U/mL), IL-12 (1000 U/mL), IL-15 (1000 U/mL), and αCD-3 antibodies (10 ng/mL) in RPMI 1640 complete medium for 14 days (medium change every two days) to expand cancer cell-specific T cells.

### (4) Cancer Cell-Specific T Cells for Cancer Treatment

Female C57BL/6 aged 6-8 weeks were selected as model mice to prepare colon cancer mice. Each mouse was subcutaneously inoculated with 2×10⁶ MC38 cells on the lower right side of the back on day 0. 0.8 million CD8⁺ cancer cell-specific T cells and 0.4 million CD4⁺ cancer cell-specific T cells were intravenously injected on day 6, day 9, day 12, day 15, day 20, and day 25 respectively after inoculation of colon cancer cells; or 1.2 million CD8⁺ cancer cell-specific T cells were injected on the above days. The methods of monitoring tumor growth and survival in the mice were the same as above.

### (5) Experimental Results

As shown in FIG. 20, compared with the control group, the tumor growth rate of the mice treated with cancer cell-specific T cells obtained by nanoparticle-assisted isolation and expansion was significantly slower and the survival time of the mice was significantly prolonged. Moreover, CD8⁺ T cells and CD4⁺ T cells obtained using nanoparticle-assisted isolation and expansion simultaneously were better than CD8⁺ T cells obtained using nanoparticle-assisted isolation and expansion alone. Furthermore, cancer cell-specific T cells obtained by assisted isolation and expansion of the nanoparticles loaded with the mixed adjuvants, lysate components and lysosome escape-enhancing substance were better than those obtained by assisted isolation and expansion of the nanoparticles loaded with the lysate components, single CpG adjuvant, and lysosome escape-enhancing substance. It can be seen that the cancer cell-specific T cells of the present disclosure had an excellent therapeutic effect on cancer.

Obviously, the above examples are merely examples for clarity of illustration, and are not limiting the implementations. For those skilled in the art, on the basis of the above description, other changes or modifications in different forms can be made. All implementations need not be and cannot be exhaustive herein. However, obvious changes or modifications derived therefrom are still within the scope of protection of the present disclosure.

1-21. (canceled)

22. A cell system derived from a tumor-infiltrating lymphocyte, comprising a cancer cell-specific T cell extracted from the tumor-infiltrating lymphocyte;
wherein the extraction comprises steps of co-incubating the tumor-infiltrating lymphocyte or a T cell in the tumor-infiltrating lymphocyte and an antigen-presenting cell with a nanoparticle loaded with a whole-cell antigen of a cancer cell and/or a microparticle loaded with the whole-cell antigen of the cancer cell to activate the cancer cell-specific T cell, and then isolating the cancer cell-specific T cell activated by the whole-cell antigen of the cancer cell,
wherein the whole-cell antigen of the cancer cell comprises a water-soluble antigen and/or a water-insoluble antigen obtained by lysing the cancer cell and/or tumor tissue, and the water-insoluble antigen is loaded onto the nanoparticle or microparticle after being solubilized by a solubilizing agent or a solubilizing solution containing the solubilizing agent.

23. The cell system according to claim 22, wherein after isolating the cancer cell-specific T cell activated by the whole-cell antigen of the cancer cell, a step of expanding the cancer cell-specific T cell or a step of expanding and sorting the cancer cell-specific T cell is further comprised.

24. The cell system according to claim 23, wherein the expanding and sorting is co-incubating the cancer cell-specific T cell with a cytokine and/or an antibody.

25. The cell system according to claim 22, wherein the isolating comprises a step of screening using a surface marker of the cancer cell-specific T cell activated by the whole-cell antigen of the cancer cell.

26. The cell system according to claim 25, wherein the surface marker comprises one or more of CD69, CD25, OX40, CD137, and CD28.

27. The cell system according to claim 22, wherein the T cell in the tumor-infiltrating lymphocyte is a T cell sorted from the tumor-infiltrating lymphocyte, and the sorting comprises sorting a CD45⁺ cell and/or a CD3⁺ cell, a CD45⁺ CD3⁺ cell, a CD3⁺ CD8⁺ cell, a CD45⁺ CD3⁺ CD8⁺ cell, a CD3⁺ CD4⁺ cell, or a CD45⁺ CD3⁺ CD4⁺ cell from the tumor-infiltrating lymphocyte.

28. The cell system according to claim 22, wherein the antigen-presenting cell comprises one or more of a B cell, a dendritic cell and a macrophage.

29. The cell system according to claim 22, wherein a cytokine is added during the co-incubating.

30. The cell system according to claim 29, wherein the cytokine comprises one or more of an interleukin, an interferon, a tumor necrosis factor, and a colony-stimulating factor.

31. The cell system according to claim 22, wherein the nanoparticle or microparticle is further loaded with an immune-enhancing adjuvant, and the immune-enhancing adjuvant comprises two or more Toll-like receptor agonists.

32. The cell system according to claim 22, wherein the nanoparticle or microparticle is further loaded with a substance that increases lysosome escape.

33. The cell system according to claim 22, wherein the solubilizing agent is selected from one or more of urea, guanidine hydrochloride, a deoxycholate, a dodecyl sulfate, glycerol, a protein-degrading enzyme, albumin, lecithin, an inorganic salt, Triton, Tween, an amino acid, a glycoside, and choline.

34. An application of the cell system according to claim 22 in preparation of a medicament for treatment or prevention of cancer.

35. The application according to claim 34, wherein the tumor-infiltrating lymphocyte or the T cell in the tumor-infiltrating lymphocyte is derived from an autologous or allogeneic source.

36. A method for activating a cancer cell-specific T cell in vitro, comprising steps of:
co-incubating a nanoparticle loaded with a whole-cell antigen of a cancer cell and/or a microparticle loaded with the whole-cell antigen of the cancer cell and an antigen-presenting cell with the cancer cell-specific T cell or a cell mixture containing the cancer cell-specific T cell,
wherein the whole-cell antigen of the cancer cell comprises a water-soluble antigen and/or a water-insoluble antigen obtained by lysing the cancer cell and/or tumor tissue, and the water-insoluble antigen is loaded onto the nanoparticle or microparticle after being solubilized by a solubilizing agent.

37. The method according to claim 36, wherein a cytokine is added during the co-incubating.

38. The method according to claim 37, wherein the cytokine comprises one or more of an interleukin, an interferon, a tumor necrosis factor, and a colony-stimulating factor.

39. The method according to claim 36, wherein the solubilizing agent is selected from one or more of urea, guanidine hydrochloride, deoxycholate, dodecyl sulfate, glycerol, a protein-degrading enzyme, albumin, lecithin, an inorganic salt, Triton, Tween, an amino acid, a glycoside, and choline.

40. The method according to claim 36, wherein the nanoparticle or microparticle is further loaded with an immune-enhancing adjuvant and/or a substance that increases lysosome escape; and the immune-enhancing adjuvant comprises two or more Toll-like receptor agonists.

41. A cancer cell-specific T cell activated in vitro by the method according to claim 22.


SEQUENCE LISTING (6 sequences)

SEQ ID NO: 1 - protein, 27 aa, Artificial Sequence
  Synthetic
FRRKAFLHWYTGEAMDEMEFTEAESNM

SEQ ID NO: 2 - protein, 27 aa, Artificial Sequence
  Synthetic
TAVITPPTTTTKKARVSTPKPATPSTD

SEQ ID NO: 3 - protein, 27 aa, Artificial Sequence
  Synthetic
NHSGLVTFQAFIDVMSRETTDTDTADQ

SEQ ID NO: 4 - protein, 9 aa, Artificial Sequence
  Synthetic
SVYDFFVWL

SEQ ID NO: 5 - protein, 30 aa, Artificial Sequence
  Synthetic
WEAKLAKALAKALAKHLAKALAKALKACEA

SEQ ID NO: 6 - protein, 8 aa, Artificial Sequence
  Synthetic
RRRRRRRR